SEQUENCE LISTING (91 sequences)

SEQ ID NO: 1 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 2 - DNA, 909 bp, synthetic construct
  source 1..909: mol_type = other DNA
gaagtggagaaaactgcgtgccctagtgggaaaaaggcgcgggaaatagacgaatcactgattttttacaaaaagtgggaactggaagcatgtgttgacgcggctctcctcgccacgcagatggaccgagtaaacgctataccgtttacgtacgaacagcttgacgtattgaagcataagctggacgaactgtatccccagggttaccctgagagcgtgattcagcaccttggatatttgttccttaaaatgtctcccgaggacattaggaaatggaatgtcacctctttggaaacactgaaggcgttgctggaggtgaataaagggcacgaaatgagtccgcaagctccaagacgcccacttccccaggtagcaacattgattgaccgattcgtcaagggacgggggcagctcgacaaagacacattggacacactcaccgcattttatccggggtatttgtgctccctcagtccagaagaactgtcatcagtaccaccctcttctatctgggctgtaagaccccaagacctcgacacatgcgatccgaggcagcttgacgtcctctaccctaaggcgaggctggcctttcagaacatgaacgggtccgagtatttcgtaaaaattcaaagttttctcggcggggcacccacagaagacctcaaagccctgtcacagcaaaacgtgagtatggacctggcaacctttatgaagttgcggaccgacgcggttctccctcttactgttgctgaggtgcaaaagctcttggggccccatgttgaaggacttaaagcagaagaacgccaccgaccggttcgagattggatacttcggcaaaggcaggatgatcttgataccttgggcctcggactgcaaggtggtattccaaatggctacttggtcctggac

SEQ ID NO: 3 - DNA, 192 bp, synthetic construct
  source 1..192: mol_type = other DNA
gaagtggagaaaacagcctgcccttctggcaaaaaagcgcgagagatcgacgaaagtttgattttctataagaagtgggaattggaagcgtgtgtagatgcagccctgctggctacgcagatggaccgggtaaacgctatcccgttcacatacgaacaattggatgtcctcaagcataaactcgatgagttg

SEQ ID NO: 4 - protein, 18 aa, synthetic construct
MALPVTALLLPLALLLHA

SEQ ID NO: 5 - DNA, 54 bp, synthetic construct
  source 1..54: mol_type = other DNA
atggctctcccagtgactgccctactgcttcccctagcgcttctcctgcatgca

SEQ ID NO: 6 - protein, 625 aa, synthetic construct
MALPTARPLLGSCGSPICSRSFLLLLLSLGWIPRLQTQTTKTSQEATLLHAVNGAADFASLPTGLFLGLTCEEVSDLSMEQAKGLAMAVRQKNITLRGHQLRCLARRLPRHLTDEELNALPLDLLLFLNPAMFPGQQACAHFFSLISKANVDVLPRRSLERQRLLMEALKCQGVYGFQVSEADVRALGGLACDLPGKFVARSSEVLLPWLAGCQGPLDQSQEKAVREVLRSGRTQYGPPSKWSVSTLDALQSLVAVLDESIVQSIPKDVKAEWLQHISRDPSRLGSKLTVIHPRFRRDAEQKACPPGKEPYKVDEDLIFYQNWELEACVDGTMLARQMDLVNEIPFTYEQLSIFKHKLDKTYPQGYPESLIQQLGHFFRYVSPEDIHQWNVTSPDTVKTLLKVSKGQKMNAQAIALVACYLRGGGQLDEDMVKALGDIPLSYLCDFSPQDLHSVPSSVMWLVGPQDLDKCSQRHLGLLYQKACSAFQNVSGLEYFEKIKTFLGGASVKDLRALSQHNVSMDIATFKRLQVDSLVGLSVAEVQKLLGPNIVDLKTEEDKSPVRDWLFRQHQKDLDRLGLGLQGGIPNGYLVLDFNVREAFSSRASLLGPGFVLIWIPALLPALRLS

SEQ ID NO: 7 - protein, 220 aa, synthetic construct
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 9 - protein, 218 aa, synthetic construct
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 10 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 11 - protein, 247 aa, synthetic construct
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 12 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 13 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 17 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 18 - protein, 13 aa, synthetic construct
EQKLISEEDLAAA

SEQ ID NO: 19 - DNA, 39 bp, synthetic construct
  source 1..39: mol_type = other DNA
gaacagaaactgatctccgaggaggacctcgcggccgca

SEQ ID NO: 20 - protein, 553 aa, synthetic construct
MALPVTALLLPLALLLHAEVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1662 bp, synthetic construct
  source 1..1662: mol_type = other DNA
atggctctcccagtgactgccctactgcttcccctagcgcttctcctgcatgcagaagtggagaaaactgcgtgccctagtgggaaaaaggcgcgggaaatagacgaatcactgattttttacaaaaagtgggaactggaagcatgtgttgacgcggctctcctcgccacgcagatggaccgagtaaacgctataccgtttacgtacgaacagcttgacgtattgaagcataagctggacgaactgtatccccagggttaccctgagagcgtgattcagcaccttggatatttgttccttaaaatgtctcccgaggacattaggaaatggaatgtcacctctttggaaacactgaaggcgttgctggaggtgaataaagggcacgaaatgagtccgcaagctccaagacgcccacttccccaggtagcaacattgattgaccgattcgtcaagggacgggggcagctcgacaaagacacattggacacactcaccgcattttatccggggtatttgtgctccctcagtccagaagaactgtcatcagtaccaccctcttctatctgggctgtaagaccccaagacctcgacacatgcgatccgaggcagcttgacgtcctctaccctaaggcgaggctggcctttcagaacatgaacgggtccgagtatttcgtaaaaattcaaagttttctcggcggggcacccacagaagacctcaaagccctgtcacagcaaaacgtgagtatggacctggcaacctttatgaagttgcggaccgacgcggttctccctcttactgttgctgaggtgcaaaagctcttggggccccatgttgaaggacttaaagcagaagaacgccaccgaccggttcgagattggatacttcggcaaaggcaggatgatcttgataccttgggcctcggactgcaaggtggtattccaaatggctacttggtcctggacgaacagaaactgatctccgaggaggacctcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 22 - protein, 314 aa, synthetic construct
MALPVTALLLPLALLLHAEVEKTACPSGKKAREIDESLIFYKKWELEACVDAALLATQMDRVNAIPFTYEQLDVLKHKLDELEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 945 bp, synthetic construct
  source 1..945: mol_type = other DNA
atggctctcccagtgactgccctactgcttcccctagcgcttctcctgcatgcagaagtggagaaaacagcctgcccttctggcaaaaaagcgcgagagatcgacgaaagtttgattttctataagaagtgggaattggaagcgtgtgtagatgcagccctgctggctacgcagatggaccgggtaaacgctatcccgttcacatacgaacaattggatgtcctcaagcataaactcgatgagttggaacagaaactgatctccgaggaggacctcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 24 - protein, 368 aa, synthetic construct
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 25 - DNA, 1104 bp, synthetic construct
  source 1..1104: mol_type = other DNA
atggtacttgaggtctccgatcatcaagttttgaacgacgctgaggtggcagctctcttggagaactttagcagtagttacgactatggagagaatgagagcgattcttgttgcacgagccccccctgtcctcaggacttctctctcaactttgatcgcgcattcctgcccgcgctctatagtttgcttttcctgcttggcctcctcggtaacggcgcagtcgctgccgttctccttagtcgacgaacggcattgtctagtacagatacgttccttcttcacctggcagtggcagacacccttttggtgttgaccttgcctctctgggcggttgatgccgctgttcagtgggtctttggtagtgggctctgtaaggtggccggcgctctttttaatatcaatttctacgcaggcgcacttctgttggcttgcatctcttttgatcgctatcttaatatagttcacgccacacagctgtatcggagaggtcccccggcacgcgtgacattgacatgtcttgcggtttggggactgtgccttctgtttgcactccccgacttcatttttctgtcagctcaccatgacgagcgcctcaacgcaacgcattgccagtataacttcccgcaagtaggtaggaccgcccttcgagttttgcagcttgttgccgggttccttctgcctctgttggttatggcgtattgttacgctcacattcttgcggtactgttggttagcaggggccaacgacggctcagggccatgagattggtggtagtcgtcgtagtagctttcgcactttgctggaccccttaccacttggtggtactcgttgacatcttgatggacctcggcgccctggcgaggaattgcggacgggagtctagagtggatgtcgccaagagtgttacgtctggcctggggtatatgcattgctgtcttaatcctctcttgtacgccttcgtgggcgttaaatttagagagagaatgtggatgctgctgcttcgcctcgggtgtcccaaccaacgagggctgcagcggcaacctagctcatcccgcagggattcctcatggagtgagacgagtgaggcgagttacagtggtctg

SEQ ID NO: 26 - protein, 415 aa, synthetic construct
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 27 - DNA, 1248 bp, synthetic construct
  source 1..1248: mol_type = other DNA
atggaactgagaaaatacgggccggggcggctcgcaggaactgtcatcggtggcgcagcacagagtaaatcacagacgaaatccgatagtatcacaaaggagtttttgcccggattgtatactgcacctagttccccatttccgccaagccaagtttccgaccatcaggtgctcaatgatgcagaggtggcagccctgctcgaaaattttagctcttcttatgattatggggaaaacgaaagtgattcttgttgcactagcccgccatgcccacaagattttagcctcaattttgatagggccttccttcccgccctgtactccctcctgtttctccttggcttgcttgggaacggagcagtagcagccgtactgctcagccggcgaactgcccttagtagcactgatacttttttgctgcaccttgctgtcgctgacacgctcctcgtgttgacactgcccctctgggcggtggatgcggccgttcagtgggtgtttggaagtggactttgcaaggttgccggcgccctctttaatatcaacttttacgcgggcgcgttgcttttggcttgtatttcatttgaccgatacttgaacattgttcacgcgacacaactctatcgaagaggaccccccgcacgcgtcacactcacttgtctcgctgtttggggcctttgtctcttgttcgccttgccagacttcattttcctttcagcgcatcacgatgaaaggctgaatgcgacgcattgtcaatataattttccccaggtagggcggaccgcgctgcgagtacttcagttggtcgctggttttttgttgcccctcttggtaatggcctactgttatgcgcatattcttgcggttctgctcgttagcagggggcaacgacgacttcgggcaatgcggttggttgtggtggtggttgttgcattcgccttgtgttggacaccctaccacctggtcgtgctcgtggacattctcatggacttgggggcgctcgctcgaaactgtggacgggaatcaagggtggatgtggctaagtcagtgacatcagggttgggatacatgcactgctgcctcaatccgctgctgtatgcctttgtcggggtgaagtttcgagaaaggatgtggatgctgcttctgcgccttggctgtcccaaccaaagagggctccaacgacagccgtcaagcagccgccgcgatagtagctggagtgaaactagtgaggcctcctactccggactgtga

SEQ ID NO: 28 - protein, 267 aa, synthetic construct
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQGSSSGSGCGCCSCAWAAPTREGSRGSHRLPAGIHPGLRPQRPPTRACEAGIRAPLSPI

SEQ ID NO: 29 - DNA, 804 bp, synthetic construct
  source 1..804: mol_type = other DNA
atggttcttgaagtcagtgatcaccaggtactcaacgatgccgaagtggctgctctgttggaaaatttttcctcctcttacgattacggtgaaaatgaaagcgatagttgttgtaccagccctccctgtccccaagacttttccttgaattttgatagagcttttctgccagctttgtattctctgctgtttctgcttgggttgttgggtaacggggccgtggccgcagtgcttctttcccgcagaaccgcacttagctctacagacacattcctcctgcacttggctgtagccgatactctcctggtattgacactccctctttgggccgtagatgctgctgtccagtgggtttttggttccggcctctgcaaagtggcaggtgctttgtttaatatcaacttttacgctggggctttgcttctcgcctgtatttcttttgatcgatacctcaacattgtgcacgctacgcaactttaccgcagggggccaccagcccgcgtcactcttacctgcctcgccgtatggggcctctgtctcctgtttgcgctgcccgacttcatatttttgtctgcacatcacgatgaacgattgaacgctacgcattgtcagtacaactttcctcaggggtcctccagtgggagcgggtgcggatgctgctcttgtgcatgggctgcaccaacaagagagggttctagaggctctcatcgacttcccgccggaatccatcccggactgcgcccccaaaggcctcctaccagggcgtgcgaagcggggattagggcacccctttcaccaatctga

SEQ ID NO: 30 - protein, 20 aa, synthetic construct
METDTLLLWVLLLWVPGSTG

SEQ ID NO: 31 - DNA, 60 bp, synthetic construct
  source 1..60: mol_type = other DNA
atggaaactgacacacttcttttgtgggttctgctcctttgggtgcctggaagcacagga

SEQ ID NO: 32 - protein, 22 aa, synthetic construct
GSGATNFSLLKQAGDVEENPGP

SEQ ID NO: 33 - DNA, 66 bp, synthetic construct
  source 1..66: mol_type = other DNA
ggatctggagcaacaaacttctcactactcaaacaagcaggtgacgtggaggagaatcccggaccc

SEQ ID NO: 34 - protein, 4 aa, synthetic construct
YMNM

SEQ ID NO: 35-36 - skipped (2 entries)

SEQ ID NO: 37 - protein, 4 aa, synthetic construct
YENV

SEQ ID NO: 38 - protein, 4 aa, synthetic construct
YSNV

SEQ ID NO: 39 - protein, 4 aa, synthetic construct
YKNL

SEQ ID NO: 40 - protein, 4 aa, synthetic construct
YENQ

SEQ ID NO: 41 - protein, 4 aa, synthetic construct
YKNI

SEQ ID NO: 42 - protein, 4 aa, synthetic construct
YINQ

SEQ ID NO: 43 - protein, 4 aa, synthetic construct
YHNK

SEQ ID NO: 44 - protein, 4 aa, synthetic construct
YVNQ

SEQ ID NO: 45 - protein, 4 aa, synthetic construct
YLNP

SEQ ID NO: 46 - protein, 4 aa, synthetic construct
YLNT

SEQ ID NO: 47 - protein, 4 aa, synthetic construct
YDND

SEQ ID NO: 48 - protein, 4 aa, synthetic construct
YENI

SEQ ID NO: 49 - protein, 4 aa, synthetic construct
YENL

SEQ ID NO: 50 - protein, 4 aa, synthetic construct
YKNQ

SEQ ID NO: 51 - protein, 4 aa, synthetic construct
YKNV

SEQ ID NO: 52 - protein, 4 aa, synthetic construct
YANG

SEQ ID NO: 53 - protein, 4 aa, synthetic construct
YMDM

SEQ ID NO: 54 - protein, 4 aa, synthetic construct
YMPM

SEQ ID NO: 55 - protein, 4 aa, synthetic construct
YMRM

SEQ ID NO: 56 - protein, 4 aa, synthetic construct
YMSM

SEQ ID NO: 57 - skipped

SEQ ID NO: 58 - protein, 4 aa, synthetic construct
YTHM

SEQ ID NO: 59 - protein, 4 aa, synthetic construct
YVLM

SEQ ID NO: 60 - protein, 4 aa, synthetic construct
YIAM

SEQ ID NO: 61 - protein, 4 aa, synthetic construct
YVEM

SEQ ID NO: 62 - protein, 4 aa, synthetic construct
YVKM

SEQ ID NO: 63 - protein, 4 aa, synthetic construct
YVPM

SEQ ID NO: 64 - skipped

SEQ ID NO: 65 - protein, 4 aa, synthetic construct
YMAP

SEQ ID NO: 66-67 - skipped (2 entries)

SEQ ID NO: 68 - protein, 4 aa, synthetic construct
YMNV

SEQ ID NO: 69 - protein, 4 aa, synthetic construct
YENM

SEQ ID NO: 70 - protein, 4 aa, synthetic construct
YMNQ

SEQ ID NO: 71 - protein, 4 aa, synthetic construct
YMNL

SEQ ID NO: 72 - protein, 4 aa, synthetic construct
YSNM

SEQ ID NO: 73 - skipped

SEQ ID NO: 74 - protein, 4 aa, synthetic construct
YGGG

SEQ ID NO: 75 - protein, 4 aa, synthetic construct
YAAA

SEQ ID NO: 76 - protein, 4 aa, synthetic construct
YFFF

SEQ ID NO: 77 - protein, 4 aa, synthetic construct
YETV

SEQ ID NO: 78 - protein, 4 aa, synthetic construct
YQQQ

SEQ ID NO: 79 - protein, 4 aa, synthetic construct
YHAE

SEQ ID NO: 80 - protein, 4 aa, synthetic construct
YLDL

SEQ ID NO: 81 - protein, 4 aa, synthetic construct
YLIP

SEQ ID NO: 82 - protein, 4 aa, synthetic construct
YLRV

SEQ ID NO: 83 - protein, 4 aa, synthetic construct
YTAV

SEQ ID NO: 84 - protein, 4 aa, synthetic construct
YVHV

SEQ ID NO: 85 - protein, 41 aa, synthetic construct
RSKRSRLLHSDYENVTPRRPGPTRKHYQPYAPPRDFAAYRS

SEQ ID NO: 86 - protein, 41 aa, synthetic construct
RSKRSRLLHSDYKNITPRRPGPTRKHYQPYAPPRDFAAYRS

SEQ ID NO: 87 - protein, 41 aa, synthetic construct
RSKRSRLLHSDYMDMTPRRPGPTRKHYQPYAPPRDFAAYRS

SEQ ID NO: 88 - protein, 41 aa, synthetic construct
RSKRSRLLHSDYGGGTPRRPGPTRKHYQPYAPPRDFAAYRS

SEQ ID NO: 89 - protein, 41 aa, synthetic construct
RSKRSRLLHSDYSNVTPRRPGPTRKHYQPYAPPRDFAAYRS

SEQ ID NO: 90 - protein, 553 aa, synthetic construct
MALPVTALLLPLALLLHAEVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDEQKLISEEDLAAAIEVMYPPPYLDNEKSNGTIIHVKGKHLCPSPLFPGPSKPFWVLVVVGGVLACYSLLVTVAFIIFWVRSKRSRLLHSDYSNVTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 1662 bp, synthetic construct
  source 1..1662: mol_type = other DNA
atggctctcccagtgactgccctactgcttcccctagcgcttctcctgcatgcagaagtggagaaaactgcgtgccctagtgggaaaaaggcgcgggaaatagacgaatcactgattttttacaaaaagtgggaactggaagcatgtgttgacgcggctctcctcgccacgcagatggaccgagtaaacgctataccgtttacgtacgaacagcttgacgtattgaagcataagctggacgaactgtatccccagggttaccctgagagcgtgattcagcaccttggatatttgttccttaaaatgtctcccgaggacattaggaaatggaatgtcacctctttggaaacactgaaggcgttgctggaggtgaataaagggcacgaaatgagtccgcaagctccaagacgcccacttccccaggtagcaacattgattgaccgattcgtcaagggacgggggcagctcgacaaagacacattggacacactcaccgcattttatccggggtatttgtgctccctcagtccagaagaactgtcatcagtaccaccctcttctatctgggctgtaagaccccaagacctcgacacatgcgatccgaggcagcttgacgtcctctaccctaaggcgaggctggcctttcagaacatgaacgggtccgagtatttcgtaaaaattcaaagttttctcggcggggcacccacagaagacctcaaagccctgtcacagcaaaacgtgagtatggacctggcaacctttatgaagttgcggaccgacgcggttctccctcttactgttgctgaggtgcaaaagctcttggggccccatgttgaaggacttaaagcagaagaacgccaccgaccggttcgagattggatacttcggcaaaggcaggatgatcttgataccttgggcctcggactgcaaggtggtattccaaatggctacttggtcctggacgaacagaaactgatctccgaggaggacctcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactactcaaatgttactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

(19) United States
(12) Patent Application Publication
(10) Pub. No.: US 2024/0189426 A1
(43) Pub. Date: Jun. 13, 2024

(54) CHIMERIC RECEPTORS TARGETING MUC16 AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Parwiz ABRAHIMI, New York, NY (US); Nan CHEN, New York, NY (US); Renier J. BRENTJENS, Short Hills, NJ (US); Jedd D. WOLCHOK, New York, NY (US); Taha MERGHOUB, Jersey City, NJ (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 18/588,764

(22) Filed: Feb. 27, 2024

Related U.S. Application Data
(63) Continuation of application No. PCT/US22/75643, filed on Aug. 30, 2022.
(60) Provisional application No. 63/238,734, filed on Aug. 30, 2021.

(51) Int. Cl. A61K 39/00; A61P 35/00; C07K 14/54; C07K 14/705; C07K 14/715; C07K 14/725; C12N 5/0783

(52) U.S. Cl. CPC A61K 39/46447; A61K 39/4611; A61K 39/4631; A61P 35/00; C07K 14/54; C07K 14/705; C07K 14/7051; C07K 14/70521; C07K 14/7151; C12N 5/0636; C07K 2319/03; C12N 2510/00

(57) ABSTRACT

The presently disclosed subject matter provides for chimeric receptors that target MUC16 and cells comprising such chimeric receptors. The presently disclosed subject matter further provides uses of the chimeric receptors for treatment.

### CROSS-REFERENCE TO RELATED APPLICATIONS

The present application is a Continuation application of International Patent Application No. PCT/US2022/075643, filed on Aug. 30, 2022, which claims priority to U.S. Provisional Patent Application No. 63/238,734, filed on Aug. 30, 2021, the contents of each of which are incorporated by reference in their entireties, and to each of which priority is claimed.

### SEQUENCE LISTING

The present application contains a Sequence Listing which has been submitted via EFS-Web and is hereby incorporated by reference in its entirety. Said Sequence Listing, created on Feb. 26, 2024, is named 072734_1573.xml and is 90,112 bytes in size.

### 1. INTRODUCTION

The presently disclosed subject matter provides methods and compositions for immunotherapies. It relates to chimeric receptors that target MUC16, cells comprising such receptors, and methods of using such cells for treatments, e.g., for treating diseases or disorders associated with MUC16.

### 2. BACKGROUND OF THE INVENTION

Cell-based immunotherapy is a therapy with curative potential for treating cancers. Immunoresponsive cells (e.g., T cells) may be modified to target tumor antigens through the introduction of genetic material coding for receptors, e.g., chimeric receptor, specific to selected antigens. Targeted T cell therapy using specific chimeric receptors has shown clinical success in treating diverse solid and hematologic malignancies.

MUC16/CA125 is a glycoprotein with limited expression in normal tissues, which is expressed on multiple cancer cell types (including ovarian, pancreatic, bladder, and lung and stomach among others), and functions to promote metastatic invasion, immune evasion, motility, and chemotherapeutic resistance. There are needs for novel therapeutic strategies to target MUC16/CA125. Further, there is an unmet need for developing strategies capable of inducing potent cancer eradication with minimal toxicity and immunogenicity.

### 3. SUMMARY OF THE INVENTION

The presently disclosed subject matter provides chimeric receptors that target MUC16. In particular, the presently disclosed subject matter provides a chimeric receptor comprising an extracellular domain, a transmembrane domain, and an intracellular signaling domain, wherein the extracellular domain comprises a mesothelin polypeptide that binds to MUC16.

In certain embodiments, the mesothelin polypeptide comprises or consists of an amino acid sequence that is at least about 80% identical to the amino acid sequence set forth in SEQ ID NO: 1 or a fragment thereof. In certain embodiments, the mesothelin polypeptide comprises or consists of an amino acid sequence that is at least about 80% identical to amino acids 296 to 598 of SEQ ID NO: 1 or a fragment thereof. In certain embodiments, the mesothelin polypeptide comprises or consists of amino acids 296 to 598 of SEQ ID NO: 1.

In certain embodiments, the mesothelin polypeptide comprises an amino acid sequence that is within Region I of human mesothelin. In certain embodiments, the mesothelin polypeptide comprises an amino acid sequence that is within Region IAB of human mesothelin. In certain embodiments, the human mesothelin comprises or consists of the amino acid sequence set forth in SEQ ID NO: 1. In certain embodiments, the Region I comprises or consists of amino acids 296 to 390 of SEQ ID NO: 1. In certain embodiments, the Region IAB comprises or consists of amino acids 296 to 359 of SEQ ID NO: 1. In certain embodiments, the mesothelin polypeptide comprises or consists of an amino acid sequence that is at least about 80% identical to the amino acid sequence of amino acids 296 to 359 of SEQ ID NO: 1 or a fragment thereof. In certain embodiments, the mesothelin polypeptide comprises or consists of amino acids 296 to 359 of SEQ ID NO: 1.

In certain embodiments, a signal peptide is covalently joined to the N-terminus of the extracellular domain. In certain embodiments, the signal peptide comprises or consists of a CD8 polypeptide. In certain embodiments, the CD8 polypeptide comprises or consists of the amino acid sequence set forth in SEQ ID NO: 4.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM polypeptide, a CTLA-4 polypeptide, a CD27 polypeptide, a CD40 polypeptide, a NKGD polypeptide, a PD-1 polypeptide, a LAG-3 polypeptide, a 2B4 polypeptide, a BTLA polypeptide, or a combination thereof. In certain embodiments, the transmembrane domain comprises a CD28 polypeptide. In certain embodiments, the CD28 polypeptide comprises or consists of amino acids 153 to 179 of SEQ ID NO: 7. In certain embodiments, the chimeric receptor further comprises a hinge/spacer region. In certain embodiments, the hinge/spacer region comprises a CD28 polypeptide. In certain embodiments, the CD28 polypeptide comprises or consists of amino acids 114 to 152 of SEQ ID NO: 7.

In certain embodiments, the intracellular signaling domain comprises a CD3ζ polypeptide. In certain embodiments, the CD3ζ polypeptide comprises or consists of the amino acid sequence set forth in SEQ ID NO: 14. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide. In certain embodiments, the CD28 polypeptide comprises or consists of amino acids 180 to 220 of SEQ ID NO: 7.

In certain embodiments, the CD28 polypeptide comprises a mutated YMNM motif. In certain embodiments, the CD28 polypeptide comprises or consists of the amino acid sequence set forth in SEQ ID NO: 85, SEQ ID NO: 86, SEQ ID NO: 87, SEQ ID NO: 88, SEQ ID NO: 89.

In certain embodiments, the chimeric receptor is recombinantly expressed or expressed from a vector.

The presently disclosed subject matter further provides cells comprising the chimeric receptors disclosed herein. In certain embodiments, the chimeric receptor is constitutively expressed on the surface of the cell.

In certain embodiments, the cell overexpresses a chemokine receptor. In certain embodiments, the cell comprises an exogenous chemokine receptor or a fragment thereof. In certain embodiments, the chemokine receptor is CXCR3. In certain embodiments, the CXCR3 is CXCR3A.

In certain embodiments, the cell comprises an exogenous Il-18 polypeptide or a fragment thereof. In certain embodiments the cell comprises a modified promoter/enhancer at an IL-18 gene locus to thereby increase IL-18 gene expression.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, a B cell, a monocyte, and a macrophage, a stem cell from which a lymphoid cell may be differentiated, a stem cell from which a myeloid cell may be differentiated, and combinations thereof. In certain embodiments, the cell is a T cell. In certain embodiments, the T cell is selected from the group consisting of a helper T cell, a cytotoxic T cell, a memory T cell, an effector memory T cell, a regulatory T cell, a tumor-infiltrating lymphocyte (TIL), a natural killer T cell, a mucosal associated invariant T cell, a γδ T cell, and combinations thereof. In certain embodiments, the T cell is a cytotoxic T cell. In certain embodiments, the stem cell is a pluripotent stem cell. In certain embodiments, the pluripotent stem cell is an embryoid stem cell or an induced pluripotent stem cell.

Furthermore, the presently disclosed subject matter provides nucleic acid molecules encoding the chimeric receptors disclosed herein. The presently disclosed subject matter also provides nucleic acid compositions comprising a first polynucleotide encoding the chimeric receptors disclosed herein and a second polynucleotide encoding the chemokine receptors disclosed herein. Additionally, the presently disclosed subject matter also provides nucleic acid compositions comprising a first polynucleotide encoding the chimeric receptors disclosed herein and a second polynucleotide encoding an IL-18 polypeptide. The presently disclosed subject matter further provides vectors comprising the nucleic acid molecules or nucleic acid compositions disclosed herein. In certain embodiments, the vector is a retroviral vector. In certain embodiments, the retroviral vector is a γ-retroviral vector or a lentiviral vector.

The presently disclosed subject matter further provides lipid nanoparticles comprising the nucleic acid molecules or nucleic acid compositions disclosed herein. The presently disclosed subject matter provides host cells expressing the nucleic acid molecules or nucleic acid compositions disclosed herein. In certain embodiments, the host cell is a T cell.

Furthermore, the presently disclosed subject matter provides compositions comprising the cells disclosed herein. In certain embodiments, the composition is a pharmaceutical composition. In certain embodiments, the composition further comprises a pharmaceutically acceptable carrier.

The presently disclosed subject matter provides methods of reducing tumor burden in a subject. In certain embodiments, the method comprises administering to the subject the cell or the composition disclosed herein. In certain embodiments, the method reduces the number of tumor cells, reduces tumor size, and/or eradicates the tumor in the subject.

The presently disclosed subject matter provides methods of increasing or lengthening survival of a subject having a tumor or a disease or disorder associated with MUC16. In certain embodiments, the method comprises comprising administering to the subject the cell or the composition disclosed herein.

The presently disclosed subject matter provides methods of treating and/or preventing a tumor or a disease or disorder associated with MUC16 in a subject. In certain embodiments, the method comprises administering to the subject the cell or the composition disclosed herein.

The presently disclosed subject matter provides cells or compositions disclosed herein for use in reducing tumor burden in a subject having a tumor, increasing or lengthening survival of a subject having a tumor, treating a tumor in a subject, or preventing a tumor in a subject.

In certain embodiments, the tumor is associated with MUC16. In certain embodiments, the tumor is a cancer. In certain embodiments, wherein the tumor is selected from the group consisting of ovarian cancer, pancreatic cancer, bladder cancer, lung cancer, stomach cancer, endometrial cancer, breast cancer, colorectal cancer, thyroid cancer, and head and neck squamous cell cancers. In certain embodiments, wherein the tumor is selected from the group consisting of ovarian cancer, pancreatic cancer, bladder cancer, lung cancer, and stomach cancer.

The presently disclosed subject matter provides a kit for reducing tumor burden in a subject, treating and/or preventing a tumor or a disease or disorder associated with MUC16 in a subject, and/or increasing or lengthening survival of a subject having a tumor or a disease or disorder associated with MUC16, comprising the cell disclosed herein. In certain embodiments, the kit further comprises written instructions for using the cell for reducing tumor burden in a subject, treating and/or preventing a tumor or a disease or disorder associated with MUC16 in a subject, and/or increasing or lengthening survival of a subject having a tumor or a disease or disorder associated with MUC16.

In certain embodiments, the tumor is associated with MUC16. In certain embodiments, the tumor is a cancer. In certain embodiments, the tumor is selected from the group consisting of ovarian cancer, pancreatic cancer, bladder cancer, lung cancer, stomach cancer, endometrial cancer, breast cancer, colorectal cancer, thyroid cancer, and head and neck squamous cell cancers. In certain embodiments, the tumor is selected from the group consisting of ovarian cancer, pancreatic cancer, bladder cancer, lung cancer, and stomach cancer. In certain embodiments, the tumor is bladder cancer.

The presently disclosed subject matter provides methods for producing cells comprising the chimeric receptors disclosed herein. In certain embodiments, the method comprises introducing into the cell a nucleic acid that encodes the chimeric receptor.

### 4. BRIEF DESCRIPTION OF THE FIGURES

The following Detailed Description, given by way of example, but not intended to limit the invention to specific embodiments described, may be understood in conjunction with the accompanying drawings.

FIG. 1 depicts an exemplified MUC16 protein.

FIGS. 2A and 2B illustrate gene expression analysis from The Cancer Genome Atlas Urothelial Bladder Carcinoma (TCGA-BLCA) collection. FIG. 2A shows a graph depicting the upregulated genes in muscle-invasive bladder cancer (MIBC). FIG. 2B illustrates the cell distribution of the upregulated genes on MIBC.

FIGS. 3A-3C depict MUC16 expression in various tissues. FIG. 3A illustrates the tissue-specific gene expression of MUC16 from the Human Protein Atlas, demonstrating limited tissue distribution of MUC16 in normal tissues. FIG. 3B demonstrates the distribution of MUC16 mRNA expression by various cancers (data from TCGA dataset, adapted from the Human Protein Atlas). FIG. 3C demonstrates two representative cases of bladder cancer that are MUC16⁺ by immunohistochemistry.

FIG. 4 illustrates bladder cancer cell line (HT1376) and ovarian cancer cell line OVCAR3 expressing full length MUC16.

FIG. 5 illustrates that monoclonal antibody anti-MUC16 (clone 4H11) did not bind to common bladder cancer cell lines, including full length MUC16⁺ HT1376.

FIG. 6 illustrates MUC16⁺ bladder cancer cell line HT1376 binding to soluble recombinant Mesothelin-Fc, but not to UM-UC3. UM-UC3 is a MUC16⁻ bladder cancer cell line.

FIGS. 7A-7L illustrate MUC16-targeted chimeric receptor constructs disclosed herein. FIG. 7A shows a chimeric antigen receptor (CAR) comprising an anti-MUC16 scFv of antibody 4H11 scFv, a CAR comprising an anti-MUC16 scFv of antibody 3A5, a presently disclosed chimeric receptor comprising an extracellular domain that comprises a mesothelin polypeptide that comprises amino acids 296 to 598 of SEQ ID NO: 1 (“Meso28z” or “Meso-28z”), and a presently disclosed chimeric receptor comprising an extracellular domain that comprises a mesothelin polypeptide that comprises amino acids 296 to 359 of SEQ ID NO: 1 (“TruncMSLN_28 z”). FIG. 7B shows vector map encoding the Meso28z. FIG. 7C shows the linearized map of the vector encoding Meso28z. FIG. 7D shows vector map encoding the TruncMSLN_282. FIG. 7E shows the linearized map of the vector encoding TruncMSLN_28 z. FIG. 7F shows a multicistronic construct including the presently disclosed Meso28z and a presently disclosed chemokine receptor CXCR3A. FIG. 7G shows vector map encoding the multicistronic construct encoding Meso28z and CXCR3A. FIG. 7H shows the linearized map of the multicistronic vector encoding Meso28z and CXCR3A. FIG. 7I shows vector map encoding the multicistronic construct encoding Meso28z and CXCR3B. FIG. 7J shows the linearized map of the multicistronic vector encoding Meso28z and CXCR3B. FIG. 7K shows vector map encoding the multicistronic construct encoding Meso28z and CXCR3alt. FIG. 7L shows the linearized map of the multicistronic vector encoding Meso28z and CXCR3alt.

FIG. 8 depicts the cytolytic activities of T cells comprising Meso282, T cells comprising 3A5-28z, T cells comprising an anti-CD19 CAR (SJC25C1-282), and untransduced T cells.

FIG. 9 illustrates that T cells comprising Meso28z showed antigen-dependent cytolytic function in vitro. For example, T cells comprising Meso28z showed cytolytic activity towards MUC16⁺ tumor cell line (HT1376) but did not show cytolytic activity towards MUC16⁻ tumor cell line (UM-UC3).

FIG. 10 depicts the production of cytokines of T cells comprising Meso-28z, T cells comprising 3A5-282, and T cells comprising SJC25C1-28z chimeric receptors produced inflammatory cytokines in co-culture with HT1376 bladder cancer cell line, while this production was reduced or abolished upon knockout of MUC16 via CRISPR/Cas9.

FIGS. 11A and 11B illustrate that soluble MUC16 competed with Mesothelin-Fc binding to HT1376 cells in vitro but did not inhibit cytolytic capacity of T cells comprising Meso-28z. FIG. 11A shows that soluble MUC16 competed with recombinant soluble Mesothelin-Fc binding to HT1376 cells in vitro. FIG. 11B shows that soluble MUC16 did not inhibit the cytolytic capacity of T cells comprising Meso-28z at 24 h.

FIG. 12 illustrates a map of MUC16TR4, a synthetic protein composed of four (4) tandem repeats, SEA modules, ectodomain, transmembrane domain and c-terminal domains of MUC16 as a model antigen.

FIG. 13 illustrates that overexpression of MUC16TR4 on MUC16⁻ UM-UC3 bladder cancer cell line was sufficient for recognition by clone OC125, which recognized full length MUC16, recombinant soluble human Mesothelin-Fc, and recombinant soluble mouse Mesothelin-His.

FIG. 14 illustrates that overexpression of MUC16TR4 on UM-UC3 was sufficient for recognition and killing by Meso-28z and 3A5-28z.

FIGS. 15A and 15B illustrate in vivo experiments using T cells comprising Meso-282. FIG. 15A shows a human orthotopic xenograft of bladder cancer cell lines. FIG. 15B shows that intravesically-delivered T cells comprising Meso-28z reduced tumor burden in a human orthotopic xenograft model.

FIGS. 16A-16C depict in vitro and in vivo activity of Meso-28z T cells. FIG. 16A shows that Meso-28z T cells effectively killed ovarian cancer cell OVCAR3 expressing MUC16 in an in vitro killing assay. FIG. 16B demonstrates that injection of Meso-28z T cells significantly prolonged the survival of NSG mice bearing ovarian cancer cell OVCAR3. FIG. 16C shows survival curves in NSG mice bearing ovarian cancer cell OVCAR3.

FIGS. 17A-17C depict in vivo activity of Meso-28z T cells. FIG. 17A shows the expression of MUC16 on primary ovarian cancer cells isolated from ovarian cancer patient's ascites. FIG. 17B shows Meso-28z T cells effectively lysed patient cancer cells in a LDH killing assay. FIG. 17C demonstrates that in an ovarian cancer PDX model, Meso-28z T cell suppressed tumor growth.

FIG. 18 shows FACS analysis of T cells representing control (untransduced, far left), transfected with Meso-28z (middle) and transfected with a multicistronic construct encoding Meso28z and CXCR3A (far right).

FIGS. 19A and 19B depict increased expression of CXCR3 upon transfection of cells with a multicistronic construct encoding Meso28z and CXCR3A. FIG. 19A shows percentage values of total cells that are CXCR3⁺. FIG. 19B shows MFI values of the CXCR3⁺ subset.

FIG. 20 shows chemotactic migration of cells expressing Meso28z and CXCR3A is enhanced with CXCR3A overexpression.

FIG. 21 illustrates in vivo experiments using T cells comprising Meso-28z delivered either intravenously (Meso IV) or intravesically (Meso Bladder). Meso-28z CAR T cells in dotted lines.

### 5. DETAILED DESCRIPTION OF THE INVENTION

The presently disclosed subject matter provides chimeric receptors that specifically target MUC16 and cells comprising such receptors. The cells can be immunoresponsive cells, e.g., genetically modified immunoresponsive cells (e.g., T cells or NK cells). The presently disclosed subject matter also provides methods of using such cells for treatments, e.g., for treating and/or preventing diseases or disorders, e.g., diseases or disorders associated with MUC16 (e.g., bladder cancer, ovarian cancer, etc.).

Non-limiting embodiments of the present disclosure are described by the present specification and Examples.

For purposes of clarity of disclosure and not by way of limitation, the detailed description is divided into the following subsections:
5.1. Definitions;
5.2. MUC16;
5.3. Chimeric Receptors;
5.4 Cells;
5.5. Nucleic Acid Compositions and Vectors;
5.6. Polypeptides and Analogs;
5.7. Formulations and Administration;
5.8. Methods of Treatment;
5.9. Kits;
5.10. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “comprises”, “comprising”, and are intended to have the broad meaning ascribed to them in U.S. Patent Law and can mean “includes”, “including” and the like.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when a chimeric receptor binds to a target protein (e.g., an antigen), a formation of an immunological synapse occurs that includes clustering of many molecules near the bound receptor (e.g., CD4 or CD8, CD3ζ/γ/δ/ε, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In certain embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography.

The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

By “receptor” is meant a polypeptide, or portion thereof, present on a cell membrane that selectively binds one or more ligand.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleotide sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

In certain embodiments, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed MUC16-targeted chimeric receptor (e.g., the extracellular domain of the chimeric receptors) comprising the amino acid sequence. Conservative modifications can include amino acid substitutions, additions and deletions. Modifications can be introduced into the extracellular domain of the presently disclosed chimeric receptors by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within an extracellular domain of the chimeric receptor can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence are altered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. In certain embodiments, the term “exogenous” encompasses any recombinant nucleic acid molecule or polypeptide expressed in a cell, such as foreign, heterologous, and over-expressed nucleic acid molecules and polypeptides. As used herein, an “exogenous nucleic acid” refers to a nucleic acid that is not present in a native wild-type cell. An exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

As used herein, the term “tumor” refers to an abnormal mass of tissue that forms when cells grow and divide more than they should or do not die when they should. Tumors include benign tumors and malignant tumors (known as “cancers”). Benign tumors may grow large but do not spread into, or invade, nearby tissues or other parts of the body. Malignant tumors can spread into, or invade, nearby tissues. They can also spread to other parts of the body through the blood and lymph systems. Tumor is also called neoplasm. In certain embodiments, the tumor is cancer. Tumor can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

### 5.2. MUC16

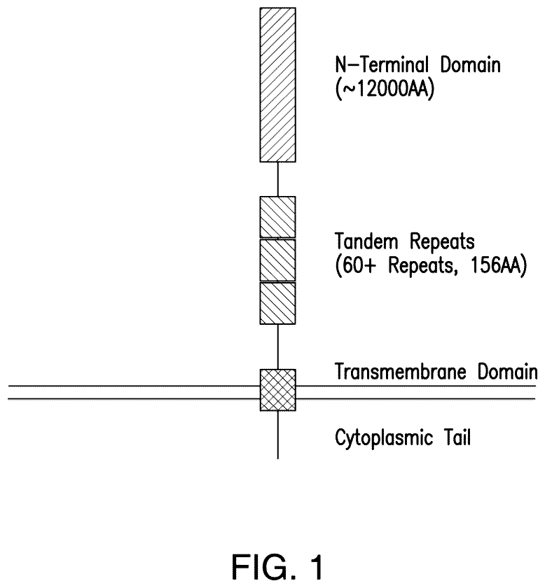

MUC16 (also known as CA125, mucin16, or cell surface associated) is a glycoprotein, and is a member of the mucin family. MUC16 is a tumor antigen widely used in monitoring cancer, e.g., ovarian cancer. MUC16 has a single transmembrane domain. MUC16 comprises about 22,000 amino acids, making it the largest membrane-associated mucin. MUC16 comprises an N-terminal domain, a tandem repeat domain, and a C-terminal domain. The N-terminal domain and tandem repeat domain are both extracellular and highly O-glycosylated. The C-terminal domain comprises multiple extracellular SEA (sea urchin sperm protein, enterokinase, and agrin) modules, a transmembrane domain, and a cytoplasmic tail. See FIG. 1.

The extracellular region of MUC16 can be released from the cell surface by undergoing proteolytic cleavage. See FIG. 1. MUC16 is thought to be cleaved at a site in the SEA modules. MUC16 can bind to mesothelin and its binding mediates heterotopic cell adhesion, which may contribute to the metastasis of cancers. For example, binding of MUC16 to mesothelin allows the metastasis of ovarian cancer to the peritoneum by initiating cell attachment to the mesothelial epithelium via binding to mesothelin.

### 5.3. Chimeric Receptors

The presently disclosed subject matter provides chimeric receptors that target or binds to MUC16. In certain embodiments, the presently disclosed chimeric receptor comprises an extracellular domain that binds to MUC16, a transmembrane domain, and an intracellular signaling domain. In certain embodiments, the presently disclosed chimeric receptor further comprises a hinge/spacer region.

### 5.3.1. Extracellular Domain

In certain embodiments, the extracellular domain binds to MUC16. In certain embodiments, the extracellular domain binds to a tandem repeat domain of MUC16.

In certain embodiments, the extracellular domain of the chimeric receptor comprises a mesothelin polypeptide. In certain embodiments, the extracellular domain of the chimeric receptor comprises a human mesothelin polypeptide.

Human mesothelin comprises a megakaryocyte potentiating factor and a GPI-anchored membrane-bound mature mesothelin. In certain embodiments, the human mesothelin comprises or consists of the amino acid sequence having a UniProt Reference No.: Q13421-1 (SEQ ID NO: 1). SEQ ID NO: 1 is provided below.

[SEQ ID NO: 1]
MALPTARPLLGSCGTPALGSLLFLLFSLGWVQPSRTLAGETGQEAAPLDG
VLANPPNISSLSPRQLLGFPCAEVSGLSTERVRELAVALAQKNVKLSTEQ
LRCLAHRLSEPPEDLDALPLDLLLFLNPDAFSGPQACTRFFSRITKANVD
LLPRGAPERQRLLPAALACWGVRGSLLSEADVRALGGLACDLPGRFVAES
AEVLLPRLVSCPGPLDQDQQEAARAALQGGGPPYGPPSTWSVSTMDALRG
LLPVLGQPIIRSIPQGIVAAWRQRSSRDPSWRQPERTILRPRFRREVEKT
ACPSGKKAREIDESLIFYKKWELEACVDAALLATQMDRVNAIPFTYEQLD
VLKHKLDELYPQGYPESVIQHLGYLFLKMSPEDIRKWNVTSLETLKALLE
VNKGHEMSPQAPRRPLPQVATLIDRFVKGRGQLDKDTLDTLTAFYPGYLC
SLSPEELSSVPPSSIWAVRPQDLDTCDPRQLDVLYPKARLAFQNMNGSEY
FVKIQSFLGGAPTEDLKALSQQNVSMDLATFMKLRTDAVLPLTVAEVQKL
LGPHVEGLKAEERHRPVRDWILRQRQDDLDTLGLGLQGGIPNGYLVLDLS
MQEALSGTPCLLGPGPVLTVLALLLASTLA

Upon proteolytic cleavage at Glu296, a cleaved mesothelin corresponding to the GPI-anchored membrane-bound mature mesothelin is obtained. In certain embodiments, the cleaved mesothelin comprises or consists of amino acids 296 to 606 of SEQ ID NO: 1. In certain embodiments, the cleaved mesothelin comprises Region I, Region II, and Region III. In certain embodiments, the Region I comprises or consists of amino acids 296 to 390 of SEQ ID NO: 1. In certain embodiments, the Region II comprises or consists of amino acids 391 to 486 of SEQ ID NO: 1. In certain embodiments, the Region III comprises or consists of amino acids 487 to 581 of SEQ ID NO: 1. In certain embodiments, Region I comprises Region IAB, and Region IBC. In certain embodiments, the Region IAB comprises or consists of amino acids 296 to 359 of SEQ ID NO: 1. In certain embodiments, the Region IBC comprises or consists of amino acids 328 to 405 of SEQ ID NO: 1. In certain embodiments, Region I comprises Region IA, Region IB, and Region IC. In certain embodiments, the Region IA comprises or consists of amino acids 296 to 337 of SEQ ID NO: 1. In certain embodiments, the Region IB comprises or consists of amino acids 328 to 369 of SEQ ID NO: 1. In certain embodiments, the Region IB comprises or consists of amino acids 360 to 405 of SEQ ID NO: 1. Details of the protein structure of mesothelin are disclosed in Kaneko et al., J Biol Chem. 2009 Feb. 6; 284(6):3739-49, the content of which is incorporated by reference.

In certain embodiments, the extracellular domain of the chimeric receptor comprises a mesothelin polypeptide that is a cleaved form of mesothelin or a fragment thereof. In certain embodiments, the extracellular domain of the chimeric receptor comprises a mesothelin polypeptide that is a cleaved form of human mesothelin or a fragment thereof.

In certain embodiments, the mesothelin polypeptide comprises or consists of an amino acid sequence that is at least about 80%, at least about 85%, about 90%, about 95%, about 96%, about 97%, about 98%, about 99% or about 100% homologous or identical to the amino acid sequence set forth in SEQ ID NO: 1 or a fragment thereof, and/or may optionally comprise up to one or up to two or up to three conservative amino acid substitutions. In certain embodiments, the mesothelin polypeptide comprises or consists of an amino acid sequence that is a consecutive portion of SEQ ID NO: 1, which is at least 50, or at least about 60, or at least about 70, or at least about 80, or at least about 90, or at least about 100, or at least about 200, or at least about 300, or at least about 400, or at least about 500, and up to 630 amino acids in length. In certain embodiments, the mesothelin polypeptide comprises or consists of amino acids 296 to 350, 296 to 359, 296 to 400, 296 to 450, 296 to 500, 296 to 550, 296 to 606, or 296 to 598 of SEQ ID NO: 1. In certain embodiments, the extracellular domain of the chimeric receptor comprises a mesothelin polypeptide comprising or consisting of amino acids 296 to 598 of SEQ ID NO: 1.

An exemplary nucleotide sequence encoding amino acids 296 to 598 of SEQ ID NO: 1 is set forth in SEQ ID NO: 2, which is provided below.

[SEQ ID NO: 2]
GAAGTGGAGAAAACTGCGTGCCCTAGTGGGAAAAAGGCGCGGGAAATAGA
CGAATCACTGATTTTTTACAAAAAGTGGGAACTGGAAGCATGTGTTGACG
CGGCTCTCCTCGCCACGCAGATGGACCGAGTAAACGCTATACCGTTTACG
TACGAACAGCTTGACGTATTGAAGCATAAGCTGGACGAACTGTATCCCCA
GGGTTACCCTGAGAGCGTGATTCAGCACCTTGGATATTTGTTCCTTAAAA
TGTCTCCCGAGGACATTAGGAAATGGAATGTCACCTCTTTGGAAACACTG
AAGGCGTTGCTGGAGGTGAATAAAGGGCACGAAATGAGTCCGCAAGCTCC
AAGACGCCCACTTCCCCAGGTAGCAACATTGATTGACCGATTCGTCAAGG
GACGGGGGCAGCTCGACAAAGACACATTGGACACACTCACCGCATTTTAT
CCGGGGTATTTGTGCTCCCTCAGTCCAGAAGAACTGTCATCAGTACCACC
CTCTTCTATCTGGGCTGTAAGACCCCAAGACCTCGACACATGCGATCCGA
GGCAGCTTGACGTCCTCTACCCTAAGGCGAGGCTGGCCTTTCAGAACATG
AACGGGTCCGAGTATTTCGTAAAAATTCAAAGTTTTCTCGGCGGGGCACC
CACAGAAGACCTCAAAGCCCTGTCACAGCAAAACGTGAGTATGGACCTGG
CAACCTTTATGAAGTTGCGGACCGACGCGGTTCTCCCTCTTACTGTTGCT
GAGGTGCAAAAGCTCTTGGGGCCCCATGTTGAAGGACTTAAAGCAGAAGA
ACGCCACCGACCGGTTCGAGATTGGATACTTCGGCAAAGGCAGGATGATC
TTGATACCTTGGGCCTCGGACTGCAAGGTGGTATTCCAAATGGCTACTTG
GTCCTGGAC

In certain embodiments, the extracellular domain of the chimeric receptor comprises a mesothelin polypeptide comprising or consisting of an amino acid sequence that is at least about 80% (e.g., at least about 85%, at least about 90%, or at least about 95%) homologous or identical to amino acids 296 to 598 of SEQ ID NO: 1. For example, the extracellular domain of the chimeric receptor comprises a mesothelin polypeptide comprising or consisting of an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to amino acids 296 to 598 of SEQ ID NO: 1. In certain embodiments, the mesothelin polypeptide comprises or consists of an amino acid sequence that is within the Region I of human mesothelin (e.g., amino acids 296 to 390 of SEQ ID NO: 1). In certain embodiments, the mesothelin polypeptide comprises or consists of an amino acid sequence that is within the Region IAB of human mesothelin (e.g., amino acids 296 to 359 of SEQ ID NO: 1). In certain embodiments, the extracellular domain of the chimeric receptor comprises a mesothelin polypeptide comprising or consisting of amino acids 296 to 359 of SEQ ID NO: 1.

An exemplary nucleotide sequence encoding amino acids 296 to 359 of SEQ ID NO: 1 is set forth in SEQ ID NO: 3, which is provided below.

[SEQ ID NO: 3]
GAAGTGGAGAAAACAGCCTGCCCTTCTGGCAAAAAAGCGCGAGAGATCGA
CGAAAGTTTGATTTTCTATAAGAAGTGGGAATTGGAAGCGTGTGTAGATG
CAGCCCTGCTGGCTACGCAGATGGACCGGGTAAACGCTATCCCGTTCACA
TACGAACAATTGGATGTCCTCAAGCATAAACTCGATGAGTTG

In certain embodiments, the extracellular domain of the chimeric receptor comprises a mesothelin polypeptide comprising or consisting of an amino acid sequence that is at least about 80% (e.g., at least about 85%, at least about 90%, or at least about 95%) homologous or identical to amino acids 296 to 359 of SEQ ID NO: 1. For example, the extracellular domain of the chimeric receptor comprises a mesothelin polypeptide comprising or consisting of an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to amino acids 296 to 359 of SEQ ID NO: 1.

In certain embodiments, the chimeric receptor comprises a leader or a signal peptide at the N-terminus of the extracellular domain. In certain embodiments, the leader or signal peptide is covalently joined to the N-terminus of the extracellular domain. In certain embodiments, the leader or a signal peptide directs the protein (e.g., the chimeric receptor) into the secretory pathway. With the signal peptide or leader, the chimeric receptor can be glycosylated and anchored in the cell membrane. The signal sequence or leader can be about 5, about 10, about 15, about 20, about 25, or about 30 amino acids in length. In certain embodiments, the leader or signal peptide comprises a CD8 polypeptide. In certain embodiments, the CD8 polypeptide comprises or consists of the amino acid sequence set forth in SEQ ID NO: 4. SEQ ID NO: 4 is provided below.

[SEQ ID NO: 4]
MALPVTALLLPLALLLHA

An exemplary nucleotide sequence encoding the amino acid sequence of SEQ ID NO: 4 is set forth in SEQ ID NO: 5, which is provided below.

[SEQ ID NO: 5]
ATGGCTCTCCCAGTGACTGCCCTACTGCTTCCCCTAGCGCTTCTCCTGCA
TGCA

In certain embodiments, the extracellular domain of the chimeric receptor comprises a murine mesothelin polypeptide. In certain embodiments, the extracellular domain of the chimeric receptor comprises a mesothelin polypeptide that is a cleaved form of murine mesothelin or a fragment thereof. In certain embodiments, the mesothelin polypeptide comprises or consists of an amino acid sequence that is at least about 85%, about 90%, about 95%, about 96%, about 97%, about 98%, about 99% or 100% homologous or identical to the amino acid sequence having a NCBI Reference No: Q61468-1 (SEQ ID NO: 6) or a fragment thereof, and/or may optionally comprise up to one or up to two or up to three conservative amino acid substitutions. In certain embodiments, the mesothelin polypeptide comprises or consists of an amino acid sequence that is a consecutive portion of SEQ ID NO: 6, which is at least about 50, or at least about 100, or at least about 200, or at least about 300, at least about 400, at least about 500, at least about 600, and up to 625 amino acids in length. In certain embodiments, the murine mesothelin polypeptide comprises or consists of amino acids 1 to 625, 1 to 600, 36 to 600, 36 to 288, 298 to 600, or 300 to 600 of SEQ ID NO: 6. In certain embodiments, the extracellular domain of the chimeric receptor comprises a mesothelin polypeptide comprising or consisting of amino acids 36 to 600 of SEQ ID NO: 6. In certain embodiments, the extracellular domain of the chimeric receptor comprises a mesothelin polypeptide comprising or consisting of amino acids 298 to 600 of SEQ ID NO: 6. SEQ ID NO: 6 is provided below:

[SEQ ID NO: 6]
MALPTARPLL GSCGSPICSR SFLLLLLSLG WIPRLQTQTT
KTSQEATLLH AVNGAADFAS LPTGLFLGLT CEEVSDLSME
QAKGLAMAVR QKNITLRGHQ LRCLARRLPR HLTDEELNAL
PLDLLLFLNP AMFPGQQACA HFFSLISKAN VDVLPRRSLE
RQRLLMEALK CQGVYGFQVS EADVRALGGL ACDLPGKFVA
RSSEVLLPWL AGCQGPLDQS QEKAVREVLR SGRTQYGPPS
KWSVSTLDAL QSLVAVLDES IVQSIPKDVK AEWLQHISRD
PSRLGSKLTV IHPRERRDAE QKACPPGKEP YKVDEDLIFY
QNWELEACVD GTMLARQMDL VNEIPFTYEQ LSIFKHKLDK
TYPQGYPESL IQQLGHFFRY VSPEDIHQWN VTSPDTVKTL
LKVSKGQKMN AQAIALVACY LRGGGQLDED MVKALGDIPL
SYLCDFSPQD LHSVPSSVMW LVGPQDLDKC SQRHLGLLYQ
KACSAFQNVS GLEYFEKIKT FLGGASVKDL RALSQHNVSM
DIATFKRLQV DSLVGLSVAE VQKLLGPNIV DLKTEEDKSP
VRDWLFRQHQ KDLDRLGLGL QGGIPNGYLV LDENVREAFS
SRASLLGPGF VLIWIPALLP ALRLS

### 5.3.2. Transmembrane Domain

In certain embodiments, the chimeric receptor comprises a transmembrane domain. In certain embodiments, the transmembrane domain is fused to the extracellular domain. In certain embodiments, the transmembrane domain is fused to the intracellular signaling domain. In certain embodiments, the transmembrane domain is positioned between the extracellular domain and the intracellular signaling domain. In certain embodiments, the transmembrane domain comprises a hydrophobic alpha helix that spans at least a portion of the membrane. Different transmembrane domains result in different receptor stability. After antigen recognition, receptors cluster and a signal are transmitted to the cell.

In certain embodiments the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM polypeptide, a CTLA-4 polypeptide, a CD27 polypeptide, a CD40 polypeptide, a NKGD polypeptide, a PD-1 polypeptide, a LAG-3 polypeptide, a 2B4 polypeptide, a BTLA polypeptide, or a combination thereof. In certain embodiments, the transmembrane domain of the chimeric receptor comprises a native or modified transmembrane domain of CD28 or a fragment thereof, a native or modified transmembrane domain of CD8 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, a native or modified transmembrane domain of NKGD2 or a fragment thereof, a native or modified transmembrane domain of PD-1 or a fragment thereof, a native or modified transmembrane domain of LAG-3 or a fragment thereof, a native or modified transmembrane domain of 2B4 or a fragment thereof, a native or modified transmembrane domain of BTLA or a fragment thereof, or a combination thereof.

In certain embodiments, the transmembrane domain of the chimeric receptor comprises a CD28 polypeptide (e.g., a transmembrane domain of CD28 or a fragment thereof).

In certain embodiments, the transmembrane domain of the chimeric receptor comprises a human CD28 polypeptide (e.g., a transmembrane domain of human CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID NO: 7) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 7, which is at least about 20, or at least about 30, or at least about 40, or at least about 50, and up to about 220 amino acids in length. In certain embodiments, the CD28 polypeptide comprises or consists of amino acids 1 to 220, 1 to 50, 50 to 100, 100 to 150, 150 to 200, 153 to 179, or 200 to 220 of SEQ ID NO: 7. In certain embodiments, the transmembrane domain of the chimeric receptor comprises a CD28 polypeptide comprising or consisting of amino acids 153 to 179 of SEQ ID NO: 7. SEQ ID NO: 7 is provided below:

| [SEQ ID NO: 7] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGENCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleotide sequence encoding amino acids 153 to 179 of SEQ ID NO: 7 is set forth in SEQ ID NO: 8, which is provided below.

[SEQ ID NO: 8]
TTTTGGGTGCTGGTGGTGGTTGGTGGAGTCCTGGCTTGCTATAGCTTGCT
AGTAACAGTGGCCTTTATTATTTTCTGGGTG

In certain embodiments, the transmembrane domain of the chimeric receptor comprises a mouse CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID NO: 9) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 9, which is at least about 20, or at least about 30, or at least about 40, or at least about 50, and up to about 218 amino acids in length. In certain embodiments, the CD28 polypeptide comprises or consists of amino acids 1 to 220, 1 to 50, 50 to 100, 100 to 150, 150 to 200, 151 to 177, or 200 to 218 of SEQ ID NO: 9. In certain embodiments, the transmembrane domain of the chimeric receptor comprises a CD28 polypeptide comprising or consisting of amino acids 151 to 177 of SEQ ID NO: 9. SEQ ID NO: 9 is provided below:

| [SEQ ID NO: 9] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In certain embodiments, the transmembrane domain of the chimeric receptor comprises a CD8 polypeptide (e.g., a transmembrane domain of CD8 or a fragment thereof).

In certain embodiments, the transmembrane domain of the chimeric receptor comprises a human CD8 polypeptide (e.g., a transmembrane domain of human CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 10) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 10, which is at least about 20, or at least about 30, or at least about 40, or at least about 50, and up to about 235 amino acids in length. In certain embodiments, the CD8 polypeptide comprises or consists of amino acids 1 to 235, 1 to 50, 50 to 100, 100 to 150, 150 to 200, 137 to 209 or 200 to 235 of SEQ ID NO: 10. In certain embodiments, the transmembrane domain of the chimeric receptor comprises a CD8 polypeptide comprising or consisting of amino acids 137 to 209 of SEQ ID NO: 10. SEQ ID NO: 10 is provided below.

[SEQ ID NO: 10]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDERRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the transmembrane domain of the chimeric receptor comprises a mouse CD8 polypeptide (e.g., a transmembrane domain of mouse CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 11) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 11, which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of amino acids 1 to 247, 1 to 50, 50 to 100, 100 to 150, 150 to 200, 151 to 219, or 200 to 247 of SEQ ID NO: 11. In certain embodiments, the transmembrane domain of the chimeric receptor comprises a CD8 polypeptide comprising or consisting of amino acids 151 to 219 of SEQ ID NO: 11. SEQ ID NO: 11 is provided below.

[SEQ ID NO: 11]
MASPLTRFLS LNLLLMGESI ILGSGEAKPQ APELRIFPKK
MDAELGQKVD LVCEVLGSVS QGCSWLFQNS SSKLPQPTEV
VYMASSHNKI TWDEKLNSSK LFSAVRDTNN KYVLTLNKES
KENEGYYFCS VISNSVMYFS SVVPVLQKVN STTTKPVLRT
PSPVHPTGTS QPQRPEDCRP RGSVKGTGLD FACDIYIWAP
LAGICVAPLL SLIITLICYH RSRKRVCKCP RPLVRQEGKP
RPSEKIV

In certain embodiments, the chimeric receptor further comprises a hinge/spacer region that links the extracellular domain to the transmembrane domain. The hinge/spacer region can be flexible enough to allow the extracellular domain to orient in different directions to facilitate antigen (e.g., MUC16) recognition while preserving the activating activity of the chimeric receptor.

In certain embodiments, the hinge/spacer region of the chimeric receptor comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 7 or SEQ ID NO: 9), a portion of a CD8 polypeptide (e.g., a portion of SEQ ID NO: 10 or SEQ ID NO: 11), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the chimeric receptor comprises a CD28 polypeptide. In certain embodiments, the hinge/spacer region of the chimeric receptor comprises a CD28 polypeptide comprising or consisting of amino acids 114 to 152 of SEQ ID NO: 7.

An exemplary nucleotide sequence encoding amino acids 114 to 152 of SEQ ID NO: 7 is set forth in SEQ ID NO: 12, which is provided below.

[SEQ ID NO: 12]
ATTGAAGTTATGTATCCTCCTCCTTACCTAGACAATGAGAAGAGCAATG
GAACCATTATCCATGTGAAAGGGAAACACCTTTGTCCAAGTCCCCTATT
TCCCGGACCTTCTAAGCCC

### 5.3.3. Intracellular Signaling Domain

In certain embodiments, the chimeric receptor comprises an intracellular signaling domain. In certain embodiments, the intracellular signaling domain of the chimeric receptor comprises a CD3ζ polypeptide. CD34 can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the chimeric receptor comprises a native CD3ζ. In certain embodiments, the native CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 13) or a fragment thereof, and/or may optionally comprise up to one or up to two or up to three conservative amino acid substitutions. In certain embodiments, the native CD3ζ polypeptide comprises or consists of an amino acid sequence that is a consecutive portion of SEQ ID NO: 13, which is at least about 20, or at least about 30, or at least about 40, or at least about 50, and up to about 164 amino acids in length. In certain embodiments, the native CD3ζ polypeptide comprises or consists of amino acids 1 to 164, 1 to 50, 50 to 100, 52 to 164, 100 to 150, or 150 to 164 of SEQ ID NO: 13. In certain embodiments, the intracellular signaling domain of the chimeric receptor comprises a CD3ζ polypeptide comprising or consisting of amino acids 52 to 164 of SEQ ID NO: 13. SEQ ID NO: 13 is provided below:

| [SEQ ID NO: 13] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the chimeric receptor comprises a CD3ζ polypeptide comprising or consisting of the amino acid sequence set forth in SEQ ID NO: 14, which is provided below.

[SEQ ID NO: 14]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

An exemplary nucleotide sequence encoding the amino acid sequence of SEQ ID NO: 14 is set forth in SEQ ID NO: 15, which is as provided below.

[SEQ ID NO: 15]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA

In certain embodiments, the intracellular signaling domain of the chimeric receptor further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than an antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, CD40, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an extracellular domain binds to its target antigen. As one example, a 4-1BB ligand (i.e., 4-1BBL) may bind to 4-1 BB for providing an intracellular signal that in combination with a chimeric receptor signal induces an effector cell function of the T cell expressing the chimeric receptor.

In certain embodiments, the intracellular signaling domain of the chimeric receptor comprises a co-stimulatory signaling region that comprises a CD28 polypeptide, e.g., an intracellular domain of CD28 or a fragment thereof. In certain embodiments, the co-stimulatory signaling region comprises a human CD28 polypeptide, e.g., an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 7 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 7, which is at least about 20, or at least about 30, or at least about 40, or at least about 50, and up to about 220 amino acids in length. In certain embodiments, the CD28 polypeptide comprises or consists of amino acids 1 to 220, 1 to 50, 50 to 100, 100 to 150, 114 to 220, 150 to 200, 180 to 220, or 200 to 220 of SEQ ID NO: 7. In certain embodiments, the intracellular signaling domain of the chimeric receptor comprises a co-stimulatory signaling region that comprises a CD28 polypeptide comprising or consisting of amino acids 180 to 220 of SEQ ID NO: 7.

An exemplary nucleotide sequence encoding amino acids 180 to 220 of SEQ ID NO: 7 is set forth in SEQ ID NO: 16, which is provided below.

[SEQ ID NO: 16]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the co-stimulatory signaling region comprises a mouse CD28 polypeptide, e.g., an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 9 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 9, which is at least about 20, or at least about 30, or at least about 40, or at least about 50, and up to about 218 amino acids in length. In certain embodiments, the CD28 polypeptide comprises or consists of amino acids 1 to 218, 1 to 50, 50 to 100, 100 to 150, 150 to 218, 178 to 218, or 200 to 218 of SEQ ID NO: 9. In certain embodiments, the co-stimulatory signaling region of a presently disclosed chimeric receptor comprises a CD28 polypeptide that comprises or consists of the amino acids 178 to 218 of SEQ ID NO: 9.

In certain embodiments, the co-stimulatory signaling region of a presently disclosed chimeric receptor comprises a CD28 polypeptide comprising a mutated YMNM motif. CD28 is a transmembrane protein that plays a critical role in T cell activation through its function as a costimulatory molecule. CD28 possesses an intracellular domain, which comprises intracellular motifs that are critical for the effective signaling of CD28. In certain embodiments, the CD28 intracellular domain comprises intracellular subdomains (also known as “intracellular motifs”) that regulate signaling pathways post TCR-stimulation. CD28 includes three intracellular motifs: a YMNM motif, and two proline-rick motifs: PRRP motif, and PYAP motif. The CD28 intracellular motifs can serve as docking sites for a number of adaptor molecules that interact with these motifs through their SH2 or SH3 domains. Such interaction transduces downstream signals terminating on transcription factors that regulate gene expression. For example, a native YMNM motif binds to a p85 subunit of a phosphoinositide 3-kinase (PI3K). A native YMNM motif also binds to growth factor receptor-bound protein 2 (Grb2) and/or Grb2-related adaptor protein 2 (GADS). Grb2 binds to Gab1 and Gab2, which in turn can recruit the p85 subunit of a PI3K.

In certain embodiments, a native YMNM motif consists of the amino acid sequence set forth in YMNM (SEQ ID NO: 34). In certain embodiments, a native YMNM motif binds to the p85 subunit of PI3K via a consensus sequence YMxM (SEQ ID NO: 35), wherein x is not an aspartic acid (N). In certain embodiments, a native YMNM motif binds to Grb2 and/or GADs via a consensus sequence YxNx (SEQ ID NO: 36), wherein x is not a methionine (M).

In certain embodiments, the CD28 polypeptide comprising a presently disclosed mutated YMNM motif has reduced recruitment of the p85 subunit of a PI3K as compared to a CD28 molecule comprising a native YMNM motif. In certain embodiments, the p85 subunit of a PI3K does not bind to the mutated YMNM motif, thereby reducing the recruitment of the p85 subunit of a PI3K to the CD28 polypeptide. The mutated YMNM motif that blocks the binding of the p85 subunit of a PI3K retains its binding to Grb2 and/or GADS. Thus, downstream signaling of Grb2/GADS remains intact, e.g., downstream signaling leading to IL-2 secretion remains intact. Such mutated YMNM motif is referred to as “GADS/Grb2-permitting mutant”.

In certain embodiments, the mutated YMNM binds to the p85 subunit of a PI3K, but does not bind to Grb2 and/or GADS. Since the binding of PI3K p85 is retained, the downstream signaling of PI3K retains intact. Since the binding of Grb2/GADS is blocked, the recruitment of PI3K p85 subunit, which is triggered by the binding of Grb2 to Gab1 and Gab2, is reduced or blocked. In addition, the downstream signaling of Grb2/GADS is blocked. Such mutated YMNM motif is referred to as “PI3K-permissive mutant”.

In certain embodiments, the mutated YMNM does not bind to the p85 subunit of a PI3K, and does not bind to Grb2 and/or GADS. Such mutated YMNM motif is referred to as “non-functional mutant”. Non-functional mutants do not provide binding of PI3K, Grb2, or GADS to CD28 at the YMNM motif, but do not preclude these signaling molecules from binding elsewhere in the CD28 molecule.

In certain embodiments, the mutated YMNM retains only one methionine residue of the two methionine residues present in the YMNM motif i.e. YMxx or YxxM. These motifs potentially modulate signaling via PI3K by limiting how many methionine residues can bind the p85 subunit of PI3K. Such mutated YMNM motif is referred to as “hybrid ‘HEMI’ mutant”.

In certain embodiments, the mutated YMNM motif is a GADS/Grb-2 permitting mutant. In certain embodiments, the mutated YMNM motif consists of the amino acid sequence set forth in YxNx (SEQ ID NO: 36), wherein x is not a methionine (M). In certain embodiments, x is selected from the group consisting of amino acids A, R, N, D, C, E, Q, G, H, I, K, F, P, S, T, W, Y, V, and L. In certain embodiments, the mutated YMNM motif consists of the amino acid sequence set forth in YENV (SEQ ID NO: 37), YSNV (SEQ ID NO: 38), YKNL (SEQ ID NO: 39), YENQ (SEQ ID NO: 40), YKNI (SEQ ID NO: 41), YINQ (SEQ ID NO: 42), YHNK (SEQ ID NO: 43), YVNQ (SEQ ID NO: 44), YLNP (SEQ ID NO: 45), YLNT (SEQ ID NO: 46), YDND (SEQ ID NO: 47), YENI (SEQ ID NO: 48), YENL (SEQ ID NO: 49), YKNQ (SEQ ID NO: 50), YKNV (SEQ ID NO: 51), or YANG (SEQ ID NO: 52). In certain embodiments, the mutated YMNM motif consists of the amino acid sequence set forth in YSNV (SEQ ID NO: 38). In certain embodiments, the mutated YMNM motif consists of the amino acid sequence set forth in YKNI (SEQ ID NO: 41). In certain embodiments, the mutated YMNM motif consists of the amino acid sequence set forth in YENV (SEQ ID NO: 37). In certain embodiments, the mutated YMNM motif consists of the amino acid sequence set forth in YKNL (SEQ ID NO: 39).

In certain embodiments, the mutated YMNM motif is a PI3K-permissive mutant. In certain embodiments, the mutated YMNM motif consists of the amino acid sequence set forth in YMxM (SEQ ID NO: 35), wherein x is not an aspartic acid (N). In certain embodiments, x is selected from the group consisting of amino acids A, R, D, C, E, Q, G, H, I, K, M, F, P, S, T, W, Y, V, and L. In certain embodiments, the mutated YMNM motif consists of the amino acid sequence set forth in YMDM (SEQ ID NO: 53), YMPM (SEQ ID NO: 54), YMRM (SEQ ID NO: 55), or YMSM (SEQ ID NO: 56). In certain embodiments, the mutated YMNM motif consists of the amino acid sequence set forth in YMDM (SEQ ID NO: 53).

In certain embodiments, the mutated YMNM motif consists of the amino acid sequence set forth in YbxM (SEQ ID NO: 57), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YTHM (SEQ ID NO: 58), YVLM (SEQ ID NO: 59), YIAM (SEQ ID NO: 60), YVEM (SEQ ID NO: 61), YVKM (SEQ ID NO: 62), or YVPM (SEQ ID NO: 63).

In certain embodiments, the mutated YMNM motif consists of the amino acid sequence set forth in YMxb (SEQ ID NO: 64), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMAP (SEQ ID NO: 65).

Certain mutated YMNM motifs are described in Mol Cell Proteomics. 2010 November; 9(11):2391-404; Virology. 2015 May; 0: 568-577, both of which are incorporated by reference herein in its entirety.

In certain embodiments, the mutated YMNM motif is a hybrid ‘HEMI’ mutant. In certain embodiments, the mutated YMNM motif consists of the amino acid sequence set forth in YMNx (SEQ ID NO: 66) or YxNM (SEQ ID NO: 67), wherein x is not a methionine (M). In certain embodiments, x is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMNV (SEQ ID NO: 68), YENM (SEQ ID NO: 69), YMNQ (SEQ ID NO: 70), YMNL (SEQ ID NO: 71), or YSNM (SEQ ID NO: 72).

In certain embodiments, the mutated YMNM motif is a non-functional mutant. In certain embodiments, the mutated YMNM motif consists of the amino acid sequence Ybxb (SEQ ID NO: 73), wherein x is not an aspartic acid (N), and b is not a methionine (M). In certain embodiments, x is selected from the group consisting of A, R, D, C, E, Q, G, H, I, K, M, F, P, S, T, W, Y, V, and L. In certain embodiments, b is selected from the group consisting of A, R, N, D, C, E, Q, G, H, I, K, F, P, S, T, W, Y, V, and L. In certain embodiments, the mutated YMNM motif consists of the amino acid sequence set forth in YGGG (SEQ ID NO: 74), YAAA (SEQ ID NO: 75), YFFF (SEQ ID NO: 76), YETV (SEQ ID NO: 77), YQQQ (SEQ ID NO: 78), YHAE (SEQ ID NO: 79), YLDL (SEQ ID NO: 80), YLIP (SEQ ID NO: 81), YLRV (SEQ ID NO: 82), YTAV (SEQ ID NO: 83), or YVHV (SEQ ID NO: 84). In certain embodiments, the mutated YMNM motif consists of the amino acid sequence set forth in YGGG (SEQ ID NO: 74).

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YENV (SEQ ID NO: 37), wherein the CD28 polypeptide comprises or consists of the amino acid sequence set forth in SEQ ID NO: 85. SEQ ID NO: 85 is provided below.

[SEQ ID NO: 85]
RSKRSRLLHSDYENVTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YKNI (SEQ ID NO: 41), wherein the CD28 polypeptide comprises or consists of the amino acid sequence set forth in SEQ ID NO: 86. SEQ ID NO: 86 is provided below.

[SEQ ID NO: 86]
RSKRSRLLHSDYKNITPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YMDM (SEQ ID NO: 53), wherein the CD28 polypeptide comprises or consists of the amino acid sequence set forth in SEQ ID NO: 87. SEQ ID NO: 87 is provided below.

[SEQ ID NO: 87]
RSKRSRLLHSDYMDMTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YGGG (SEQ ID NO: 74), wherein the CD28 polypeptide comprises or consists of the amino acid sequence set forth in SEQ ID NO: 88. SEQ ID NO: 88 is provided below.

[SEQ ID NO: 88]
RSKRSRLLHSDYGGGTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YSNV (SEQ ID NO: 38), wherein the CD28 polypeptide comprises or consists of the amino acid sequence set forth in SEQ ID NO: 89. SEQ ID NO: 89 is provided below.

[SEQ ID NO: 89]
RSKRSRLLHSDYSNVTPRRPGPTRKHYQPYAPPRDFAAYRS

In certain embodiments, the intracellular signaling domain of the presently disclosed chimeric receptor comprises a first co-stimulatory signaling domain that comprises a CD28 polypeptide comprising a mutated YMNM motif (as disclosed herein), and a second co-stimulatory signaling domain that comprises an intracellular domain of a co-stimulatory molecule. Additional information regarding chimeric receptors including CD28 polypeptide comprising a mutated YMNM motif can be found in International Patent Publication No. WO 2021/158850, which is incorporated by reference in its entirety.

In certain embodiments, the intracellular signaling domain of the chimeric receptor comprises a co-stimulatory signaling region that comprises a 4-1BB polypeptide, e.g., an intracellular domain of 4-1BB or a fragment thereof (e.g., an intracellular domain of human 4-1BB or a fragment thereof). In certain embodiments, the co-stimulatory signaling region comprises a human 4-1BB polypeptide, e.g., an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 17) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 17, which is at least about 20, or at least about 30, or at least about 40, or at least about 50, or at least about 100, or at least about 150, or at least about 150, and up to about 255 amino acids in length. In certain embodiments, the 4-1BB polypeptide comprises or consists of amino acids 1 to 255, 1 to 50, 50 to 100, 100 to 150, 150 to 200, or 200 to 255 of SEQ ID NO: 17. In certain embodiments, the intracellular signaling domain of the chimeric receptor comprises a co-stimulatory signaling region that comprises a 4-1BB polypeptide comprising or consisting of amino acids 214 to 255 of SEQ ID NO: 17. SEQ ID NO: 17 is provided below.

| [SEQ ID NO: 17] | |
|---|---|
| 1 | MGNSCYNIVA TLLLVLNFER TRSLQDPCSN CPAGTFCDNN RNQICSPCPP NSESSAGGQR |
| 61 | TCDICRQCKG VFRTRKECSS TSNAECDCTP GFHCLGAGCS MCEQDCKQGQ ELTKKGCKDC |
| 121 | CFGTENDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In certain embodiments, the intracellular signaling domain of the chimeric receptor comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

### 5.3.4. Exemplified Chimeric Receptors

In certain embodiments, the chimeric receptor comprises (a) an extracellular domain that comprises a mesothelin polypeptide comprising or consisting of amino acids 296 to 598 of SEQ ID NO: 1, (b) a hinge/spacer region that comprises a CD28 polypeptide, (c) a transmembrane domain that comprises a CD28 polypeptide; and (d) an intracellular signaling domain that comprises (i) a CD3ζ polypeptide, and (ii) a co-stimulatory signaling region that comprises a CD28 polypeptide. In certain embodiments, the hinge/spacer region comprises a CD28 polypeptide comprising or consisting of amino acids 114 to 152 of SEQ ID NO: 7. In certain embodiments, the transmembrane domain comprises a CD28 polypeptide comprising or consisting of amino acids 153 to 179 of SEQ ID NO: 7. In certain embodiments, the CD3ζ polypeptide comprises or consists of the amino acid sequence set forth in SEQ ID NO: 14. In certain embodiments, the co-stimulatory signaling region comprises a CD28 polypeptide comprising or consisting of amino acids 180 to 220 of SEQ ID NO: 7. In certain embodiments, the chimeric receptor is designed as “Meso28z”. In certain embodiments, the chimeric receptor further comprises a CD8 signal peptide. In certain embodiments, the CD8 signal peptide comprises or consists of the amino acid sequence set forth in SEQ ID NO: 4. In certain embodiments, the chimeric receptor further comprises a Myc-AAA linker. In certain embodiments, the Myc-AAA linker comprises or consists of the amino acid sequence set forth in SEQ ID NO: 18. An exemplary nucleotide sequence the amino acid sequence of SEQ ID NO: 18 is set forth in SEQ ID NO: 19. In certain embodiments, the chimeric receptor comprises the amino acid sequence set forth in SEQ ID NO: 20.

An exemplary nucleotide sequence encoding the amino acid sequence of SEQ ID NO: 20 is set forth in SEQ ID NO: 21. SEQ ID Nos: 18-21 are provided below.

[SEQ ID NO: 18]
EQKLISEEDLAAA
[SEQ ID NO: 19]
GAACAGAAACTGATCTCCGAGGAGGACCTCGCGGCCGCA
[SEQ ID NO: 20]
MALPVTALLLPLALLLHAEVEKTACPSGKKAREIDESLIFYKKWELEAC
VDAALLATQMDRVNAIPFTYEQLDVLKHKLDELYPQGYPESVIQHLGYL
FLKMSPEDIRKWNVTSLETLKALLEVNKGHEMSPQAPRRPLPQVATLID
RFVKGRGQLDKDTLDTLTAFYPGYLCSLSPEELSSVPPSSIWAVRPQDL
DTCDPRQLDVLYPKARLAFQNMNGSEYFVKIQSFLGGAPTEDLKALSQQ
NVSMDLATFMKLRTDAVLPLTVAEVQKLLGPHVEGLKAEERHRPVRDWI
LRQRQDDLDTLGLGLQGGIPNGYLVLDEQKLISEEDLAAAIEVMYPPPY
LDNEKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAAYRS
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
[SEQ ID NO: 21]
ATGGCTCTCCCAGTGACTGCCCTACTGCTTCCCCTAGCGCTTCTCCTGC
ATGCAGAAGTGGAGAAAACTGCGTGCCCTAGTGGGAAAAAGGCGCGGGA
AATAGACGAATCACTGATTTTTTACAAAAAGTGGGAACTGGAAGCATGT
GTTGACGCGGCTCTCCTCGCCACGCAGATGGACCGAGTAAACGCTATAC
CGTTTACGTACGAACAGCTTGACGTATTGAAGCATAAGCTGGACGAACT
GTATCCCCAGGGTTACCCTGAGAGCGTGATTCAGCACCTTGGATATTTG
TTCCTTAAAATGTCTCCCGAGGACATTAGGAAATGGAATGTCACCTCTT
TGGAAACACTGAAGGCGTTGCTGGAGGTGAATAAAGGGCACGAAATGAG
TCCGCAAGCTCCAAGACGCCCACTTCCCCAGGTAGCAACATTGATTGAC
CGATTCGTCAAGGGACGGGGGCAGCTCGACAAAGACACATTGGACACAC
TCACCGCATTTTATCCGGGGTATTTGTGCTCCCTCAGTCCAGAAGAACT
GTCATCAGTACCACCCTCTTCTATCTGGGCTGTAAGACCCCAAGACCTC
GACACATGCGATCCGAGGCAGCTTGACGTCCTCTACCCTAAGGCGAGGC
TGGCCTTTCAGAACATGAACGGGTCCGAGTATTTCGTAAAAATTCAAAG
TTTTCTCGGCGGGGCACCCACAGAAGACCTCAAAGCCCTGTCACAGCAA
AACGTGAGTATGGACCTGGCAACCTTTATGAAGTTGCGGACCGACGCGG
TTCTCCCTCTTACTGTTGCTGAGGTGCAAAAGCTCTTGGGGCCCCATGT
TGAAGGACTTAAAGCAGAAGAACGCCACCGACCGGTTCGAGATTGGATA
CTTCGGCAAAGGCAGGATGATCTTGATACCTTGGGCCTCGGACTGCAAG
GTGGTATTCCAAATGGCTACTTGGTCCTGGACGAACAGAAACTGATCTC
CGAGGAGGACCTCGCGGCCGCAATTGAAGTTATGTATCCTCCTCCTTAC
CTAGACAATGAGAAGAGCAATGGAACCATTATCCATGTGAAAGGGAAAC
ACCTTTGTCCAAGTCCCCTATTTCCCGGACCTTCTAAGCCCTTTTGGGT
GCTGGTGGTGGTTGGTGGAGTCCTGGCTTGCTATAGCTTGCTAGTAACA
GTGGCCTTTATTATTTTCTGGGTGAGGAGTAAGAGGAGCAGGCTCCTGC
ACAGTGACTACATGAACATGACTCCCCGCCGCCCCGGGCCCACCCGCAA
GCATTACCAGCCCTATGCCCCACCACGCGACTTCGCAGCCTATCGCTCC
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA

In certain embodiments, the chimeric receptor comprises (a) an extracellular domain that comprises a mesothelin polypeptide comprising or consisting of amino acids 296 to 359 of SEQ ID NO: 1, (b) a hinge/spacer region that comprises a CD28 polypeptide, (c) a transmembrane domain that comprises a CD28 polypeptide; and (d) an intracellular signaling domain that comprises (i) a CD3ζ polypeptide, and (ii) a co-stimulatory signaling region that comprises a CD28 polypeptide. In certain embodiments, the hinge/spacer region comprises a CD28 polypeptide comprising or consisting of amino acids 114 to 152 of SEQ ID NO: 7. In certain embodiments, the transmembrane domain comprises a CD28 polypeptide comprising or consisting of amino acids 153 to 179 of SEQ ID NO: 7. In certain embodiments, the CD3ζ polypeptide comprises or consists of the amino acid sequence set forth in SEQ ID NO: 14. In certain embodiments, the co-stimulatory signaling region comprises a CD28 polypeptide comprising or consisting of amino acids 180 to 220 of SEQ ID NO: 7. In certain embodiments, the chimeric receptor is designed as “TruncMSLN_28 z”. In certain embodiments, the chimeric receptor further comprises a CD8 signal peptide. In certain embodiments, the CD8 signal peptide comprises or consists of the amino acid sequence set forth in SEQ ID NO: 4. In certain embodiments, the chimeric receptor further comprises a Myc-AAA linker. In certain embodiments, the Myc-AAA linker comprises or consists of the amino acid sequence set forth in SEQ ID NO: 18. In certain embodiments, the chimeric receptor comprises the amino acid sequence set forth in SEQ ID NO: 22, which is provided below.

[SEQ ID NO: 22]
MALPVTALLLPLALLLHAEVEKTACPSGKKAREIDESLIFYKKWELEAC
VDAALLATQMDRVNAIPFTYEQLDVLKHKLDELEQKLISEEDLAAAIEV
MYPPPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAPPRD
FAAYRSRVKESRSADAPAYQQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPR

An exemplary nucleotide sequence the amino acid sequence of SEQ ID NO: 22 is set forth in SEQ ID NO: 23, which is provided below.

[SEQ ID NO: 23]
ATGGCTCTCCCAGTGACTGCCCTACTGCTTCCCCTAGCGCTTCTCCTGC
ATGCAGAAGTGGAGAAAACAGCCTGCCCTTCTGGCAAAAAAGCGCGAGA
GATCGACGAAAGTTTGATTTTCTATAAGAAGTGGGAATTGGAAGCGTGT
GTAGATGCAGCCCTGCTGGCTACGCAGATGGACCGGGTAAACGCTATCC
CGTTCACATACGAACAATTGGATGTCCTCAAGCATAAACTCGATGAGTT
GGAACAGAAACTGATCTCCGAGGAGGACCTCGCGGCCGCAATTGAAGTT
ATGTATCCTCCTCCTTACCTAGACAATGAGAAGAGCAATGGAACCATTA
TCCATGTGAAAGGGAAACACCTTTGTCCAAGTCCCCTATTTCCCGGACC
TTCTAAGCCCTTTTGGGTGCTGGTGGTGGTTGGTGGAGTCCTGGCTTGC
TATAGCTTGCTAGTAACAGTGGCCTTTATTATTTTCTGGGTGAGGAGTA
AGAGGAGCAGGCTCCTGCACAGTGACTACATGAACATGACTCCCCGCCG
CCCCGGGCCCACCCGCAAGCATTACCAGCCCTATGCCCCACCACGCGAC
TTCGCAGCCTATCGCTCCAGAGTGAAGTTCAGCAGGAGCGCAGACGCCC
CCGCGTACCAGCAGGGCCAGAACCAGCTCTATAACGAGCTCAATCTAGG
ACGAAGAGAGGAGTACGATGTTTTGGACAAGAGACGTGGCCGGGACCCT
GAGATGGGGGGAAAGCCGAGAAGGAAGAACCCTCAGGAAGGCCTGTACA
ATGAACTGCAGAAAGATAAGATGGCGGAGGCCTACAGTGAGATTGGGAT
GAAAGGCGAGCGCCGGAGGGGCAAGGGGCACGATGGCCTTTACCAGGGT
CTCAGTACAGCCACCAAGGACACCTACGACGCCCTTCACATGCAGGCCC
TGCCCCCTCGCTAA

In certain embodiments, the chimeric receptor comprises (a) an extracellular domain that comprises a mesothelin polypeptide comprising or consisting of amino acids 296 to 598 of SEQ ID NO: 1, (b) a hinge/spacer region that comprises a CD28 polypeptide, (c) a transmembrane domain that comprises a CD28 polypeptide; and (d) an intracellular signaling domain that comprises (i) a CD3ζ polypeptide, and (ii) a co-stimulatory signaling region that comprises a CD28 polypeptide comprising a mutated YMNM motif consisting of the amino acid sequence set forth in YSNV (SEQ ID NO: 38). In certain embodiments, the hinge/spacer region comprises a CD28 polypeptide comprising or consisting of amino acids 114 to 152 of SEQ ID NO: 7. In certain embodiments, the transmembrane domain comprises a CD28 polypeptide comprising or consisting of amino acids 153 to 179 of SEQ ID NO: 7. In certain embodiments, the CD3ζ polypeptide comprises or consists of the amino acid sequence set forth in SEQ ID NO: 14. In certain embodiments, the co-stimulatory signaling region comprises a CD28 polypeptide comprises or consists of the amino acid sequence set forth in SEQ ID NO: 89. In certain embodiments, the chimeric receptor is designed as “Meso28zYSNV”. In certain embodiments, the chimeric receptor further comprises a CD8 signal peptide. In certain embodiments, the CD8 signal peptide comprises or consists of the amino acid sequence set forth in SEQ ID NO: 4. In certain embodiments, the chimeric receptor further comprises a Myc-AAA linker. In certain embodiments, the Myc-AAA linker comprises or consists of the amino acid sequence set forth in SEQ ID NO: 18. In certain embodiments, the chimeric receptor comprises the amino acid sequence set forth in SEQ ID NO: 90, which is provided below.

[SEQ ID NO: 90]
MALPVTALLLPLALLLHAEVEKTACPSGKKAREIDESLIFYKKWELEAC
VDAALLATQMDRVNAIPFTYEQLDVLKHKLDELYPQGYPESVIQHLGYL
FLKMSPEDIRKWNVTSLETLKALLEVNKGHEMSPQAPRRPLPQVATLID
RFVKGRGQLDKDTLDTLTAFYPGYLCSLSPEELSSVPPSSIWAVRPQDL
DTCDPRQLDVLYPKARLAFQNMNGSEYFVKIQSFLGGAPTEDLKALSQQ
NVSMDLATFMKLRTDAVLPLTVAEVQKLLGPHVEGLKAEERHRPVRDWI
LRQRQDDLDTLGLGLQGGIPNGYLVLDEQKLISEEDLAAAIEVMYPPPY
LDNEKSNGTIIHVKGKHLCPSPLFPGPSKPFWVLVVVGGVLACYSLLVT
VAFIIFWVRSKRSRLLHSDYSNVTPRRPGPTRKHYQPYAPPRDFAAYRS
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

An exemplary nucleotide sequence encoding the amino acid sequence of SEQ ID NO: 90 is set forth in SEQ ID NO: 91. SEQ ID NO: 91 is provided below.

[SEQ ID NO: 91]
ATGGCTCTCCCAGTGACTGCCCTACTGCTTCCCCTAGCGCTTCTCCTGC
ATGCAGAAGTGGAGAAAACTGCGTGCCCTAGTGGGAAAAAGGCGCGGGA
AATAGACGAATCACTGATTTTTTACAAAAAGTGGGAACTGGAAGCATGT
GTTGACGCGGCTCTCCTCGCCACGCAGATGGACCGAGTAAACGCTATAC
CGTTTACGTACGAACAGCTTGACGTATTGAAGCATAAGCTGGACGAACT
GTATCCCCAGGGTTACCCTGAGAGCGTGATTCAGCACCTTGGATATTTG
TTCCTTAAAATGTCTCCCGAGGACATTAGGAAATGGAATGTCACCTCTT
TGGAAACACTGAAGGCGTTGCTGGAGGTGAATAAAGGGCACGAAATGAG
TCCGCAAGCTCCAAGACGCCCACTTCCCCAGGTAGCAACATTGATTGAC
CGATTCGTCAAGGGACGGGGGCAGCTCGACAAAGACACATTGGACACAC
TCACCGCATTTTATCCGGGGTATTTGTGCTCCCTCAGTCCAGAAGAACT
GTCATCAGTACCACCCTCTTCTATCTGGGCTGTAAGACCCCAAGACCTC
GACACATGCGATCCGAGGCAGCTTGACGTCCTCTACCCTAAGGCGAGGC
TGGCCTTTCAGAACATGAACGGGTCCGAGTATTTCGTAAAAATTCAAAG
TTTTCTCGGCGGGGCACCCACAGAAGACCTCAAAGCCCTGTCACAGCAA
AACGTGAGTATGGACCTGGCAACCTTTATGAAGTTGCGGACCGACGCGG
TTCTCCCTCTTACTGTTGCTGAGGTGCAAAAGCTCTTGGGGCCCCATGT
TGAAGGACTTAAAGCAGAAGAACGCCACCGACCGGTTCGAGATTGGATA
CTTCGGCAAAGGCAGGATGATCTTGATACCTTGGGCCTCGGACTGCAAG
GTGGTATTCCAAATGGCTACTTGGTCCTGGACGAACAGAAACTGATCTC
CGAGGAGGACCTCGCGGCCGCAATTGAAGTTATGTATCCTCCTCCTTAC
CTAGACAATGAGAAGAGCAATGGAACCATTATCCATGTGAAAGGGAAAC
ACCTTTGTCCAAGTCCCCTATTTCCCGGACCTTCTAAGCCCTTTTGGGT
GCTGGTGGTGGTTGGTGGAGTCCTGGCTTGCTATAGCTTGCTAGTAACA
GTGGCCTTTATTATTTTCTGGGTGAGGAGTAAGAGGAGCAGGCTCCTGC
ACAGTGACTACTCAAATGTTACTCCCCGCCGCCCCGGGCCCACCCGCAA
GCATTACCAGCCCTATGCCCCACCACGCGACTTCGCAGCCTATCGCTCC
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAG

### 5.4. Cells

The presently disclosed subject matter provides cells comprising a presently disclosed chimeric receptor (e.g., one disclosed in Section 5.3). In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell. In certain embodiments, the stem cell is a human stem cell. In certain embodiments, the stem cell is a human pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of a chimeric receptor disclosed herein. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express chimeric receptors), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred chimeric receptor modified T cells upon encounter with targeted tumor cells.

In certain embodiments, the cells can be transduced with the presently disclosed chimeric receptor such that the cells express the chimeric receptor.

In certain embodiments, the presently disclosed cell overexpresses a chemokine receptor. Chemokine receptors are cytokine receptors found on the surface of certain cells that interact with a type of cytokine called a chemokine. There have been 20 distinct chemokine receptors discovered in humans. Each chemokine receptor has a rhodopsin-like 7-transmembrane (7TM) structure and couples to G-protein for signal transduction within a cell, making them members of a large protein family of G protein-coupled receptors. Upon interaction with their specific chemokine ligands, chemokine receptors trigger an intracellular calcium signaling causing several responses, including, for example, the onset of chemotaxis that traffics the cell to a desired location (e.g., tumor).

In certain embodiments, the presently disclosed cell comprises an exogenous chemokine receptor or a fragment thereof. In certain embodiments, the chemokine receptor is selected from the group consisting of a CXC chemokine receptor, a CC chemokine receptor, a CX3C chemokine receptor, or a XC chemokine receptor. In certain embodiments, the chemokine receptor is a CXC chemokine receptor. In certain embodiments, the chemokine receptor is selected from the group consisting of CXCR1, CXCR2, CXCR3, CXCR4, CXCR5, CXCR6, CXCR7, CCR1, CCR2, CCRL2, CCR3, CCR4, CCR5, CCR6, CCR7, CCR8, CCR9, CCR10, CCR11, XCR1, CX3CR1, CCBP2, and CMKLR1. In certain embodiments, the chemokine receptor binds to CXCL9, CXCL10, CXCL11, or a combination thereof. In certain embodiments, the chemokine receptor is CXCR3.

Chemokine receptor CXCR3 (also known as CD182, CD183, CKR-L2, CMKAR3, GPR9, IP10-R, Mig-R, MigR, C—X—C motif chemokine receptor 3) is a Gαi protein-coupled receptor in the CXC chemokine receptor family. There are three isoforms of CXCR3 in humans: CXCR3-A, CXCR3-B and chemokine receptor 3-alternative (CXCR3-alt). CXCR3-A binds to the CXC chemokines CXCL9 (MIG), CXCL10 (IP-10), and CXCL11 (I-TAC) whereas CXCR3-B can also bind to CXCL4 in addition to CXCL9, CXCL10, and CXCL11.

In certain embodiments, the presently disclosed cell further comprises an exogenous CXCR3 polypeptide. In certain embodiments, the exogenous CXCR3 polypeptide comprises or consists of the amino acid sequence set forth in SEQ ID NO: 24. An exemplary nucleotide sequence encoding the amino acid sequence of SEQ ID NO: 24 is set forth in SEQ ID NO: 25. SEQ ID NOS: 24 and 25 are provided below.

[SEQ ID NO: 24]
MVLEVSDHQVLNDAEVAALLENFSSSYDYGENESDSCCTSPPCPQDESL
NFDRAFLPALYSLLFLLGLLGNGAVAAVLLSRRTALSSTDTFLLHLAVA
DTLLVLTLPLWAVDAAVQWVFGSGLCKVAGALFNINFYAGALLLACISF
DRYLNIVHATQLYRRGPPARVTLTCLAVWGLCLLFALPDFIFLSAHHDE
RLNATHCQYNFPQVGRTALRVLQLVAGFLLPLLVMAYCYAHILAVLLVS
RGQRRLRAMRLVVVVVVAFALCWTPYHLVVLVDILMDLGALARNCGRES
RVDVAKSVTSGLGYMHCCLNPLLYAFVGVKFRERMWMLLLRLGCPNQRG
LQRQPSSSRRDSSWSETSEASYSGL
[SEQ ID NO: 25]
ATGGTACTTGAGGTCTCCGATCATCAAGTTTTGAACGACGCTGAGGTGG
CAGCTCTCTTGGAGAACTTTAGCAGTAGTTACGACTATGGAGAGAATGA
GAGCGATTCTTGTTGCACGAGCCCCCCCTGTCCTCAGGACTTCTCTCTC
AACTTTGATCGCGCATTCCTGCCCGCGCTCTATAGTTTGCTTTTCCTGC
TTGGCCTCCTCGGTAACGGCGCAGTCGCTGCCGTTCTCCTTAGTCGACG
AACGGCATTGTCTAGTACAGATACGTTCCTTCTTCACCTGGCAGTGGCA
GACACCCTTTTGGTGTTGACCTTGCCTCTCTGGGCGGTTGATGCCGCTG
TTCAGTGGGTCTTTGGTAGTGGGCTCTGTAAGGTGGCCGGCGCTCTTTT
TAATATCAATTTCTACGCAGGCGCACTTCTGTTGGCTTGCATCTCTTTT
GATCGCTATCTTAATATAGTTCACGCCACACAGCTGTATCGGAGAGGTC
CCCCGGCACGCGTGACATTGACATGTCTTGCGGTTTGGGGACTGTGCCT
TCTGTTTGCACTCCCCGACTTCATTTTTCTGTCAGCTCACCATGACGAG
CGCCTCAACGCAACGCATTGCCAGTATAACTTCCCGCAAGTAGGTAGGA
CCGCCCTTCGAGTTTTGCAGCTTGTTGCCGGGTTCCTTCTGCCTCTGTT
GGTTATGGCGTATTGTTACGCTCACATTCTTGCGGTACTGTTGGTTAGC
AGGGGCCAACGACGGCTCAGGGCCATGAGATTGGTGGTAGTCGTCGTAG
TAGCTTTCGCACTTTGCTGGACCCCTTACCACTTGGTGGTACTCGTTGA
CATCTTGATGGACCTCGGCGCCCTGGCGAGGAATTGCGGACGGGAGTCT
AGAGTGGATGTCGCCAAGAGTGTTACGTCTGGCCTGGGGTATATGCATT
GCTGTCTTAATCCTCTCTTGTACGCCTTCGTGGGCGTTAAATTTAGAGA
GAGAATGTGGATGCTGCTGCTTCGCCTCGGGTGTCCCAACCAACGAGGG
CTGCAGCGGCAACCTAGCTCATCCCGCAGGGATTCCTCATGGAGTGAGA
CGAGTGAGGCGAGTTACAGTGGTCTG

In certain embodiments, the exogenous CXCR3 polypeptide comprises or consists of the amino acid sequence set forth in SEQ ID NO: 26. An exemplary nucleotide sequence encoding the amino acid sequence of SEQ ID NO: 26 is set forth in SEQ ID NO: 27. SEQ ID NOS: 26 and 27 are provided below.

[SEQ ID NO: 26]
MELRKYGPGRLAGTVIGGAAQSKSQTKSDSITKEFLPGLYTAPSSPFPP
SQVSDHQVLNDAEVAALLENFSSSYDYGENESDSCCTSPPCPQDESLNF
DRAFLPALYSLLFLLGLLGNGAVAAVLLSRRTALSSTDTFLLHLAVADT
LLVLTLPLWAVDAAVQWVFGSGLCKVAGALFNINFYAGALLLACISFDR
YLNIVHATQLYRRGPPARVTLTCLAVWGLCLLFALPDFIFLSAHHDERL
NATHCQYNFPQVGRTALRVLQLVAGFLLPLLVMAYCYAHILAVLLVSRG
QRRLRAMRLVVVVVVAFALCWTPYHLVVLVDILMDLGALARNCGRESRV
DVAKSVTSGLGYMHCCLNPLLYAFVGVKFRERMWMLLLRLGCPNQRGLQ
RQPSSSRRDSSWSETSEASYSGL
[SEQ ID NO: 27]
ATGGAACTGAGAAAATACGGGCCGGGGCGGCTCGCAGGAACTGTCATCG
GTGGCGCAGCACAGAGTAAATCACAGACGAAATCCGATAGTATCACAAA
GGAGTTTTTGCCCGGATTGTATACTGCACCTAGTTCCCCATTTCCGCCA
AGCCAAGTTTCCGACCATCAGGTGCTCAATGATGCAGAGGTGGCAGCCC
TGCTCGAAAATTTTAGCTCTTCTTATGATTATGGGGAAAACGAAAGTGA
TTCTTGTTGCACTAGCCCGCCATGCCCACAAGATTTTAGCCTCAATTTT
GATAGGGCCTTCCTTCCCGCCCTGTACTCCCTCCTGTTTCTCCTTGGCT
TGCTTGGGAACGGAGCAGTAGCAGCCGTACTGCTCAGCCGGCGAACTGC
CCTTAGTAGCACTGATACTTTTTTGCTGCACCTTGCTGTCGCTGACACG
CTCCTCGTGTTGACACTGCCCCTCTGGGCGGTGGATGCGGCCGTTCAGT
GGGTGTTTGGAAGTGGACTTTGCAAGGTTGCCGGCGCCCTCTTTAATAT
CAACTTTTACGCGGGCGCGTTGCTTTTGGCTTGTATTTCATTTGACCGA
TACTTGAACATTGTTCACGCGACACAACTCTATCGAAGAGGACCCCCCG
CACGCGTCACACTCACTTGTCTCGCTGTTTGGGGCCTTTGTCTCTTGTT
CGCCTTGCCAGACTTCATTTTCCTTTCAGCGCATCACGATGAAAGGCTG
AATGCGACGCATTGTCAATATAATTTTCCCCAGGTAGGGCGGACCGCGC
TGCGAGTACTTCAGTTGGTCGCTGGTTTTTTGTTGCCCCTCTTGGTAAT
GGCCTACTGTTATGCGCATATTCTTGCGGTTCTGCTCGTTAGCAGGGGG
CAACGACGACTTCGGGCAATGCGGTTGGTTGTGGTGGTGGTTGTTGCAT
TCGCCTTGTGTTGGACACCCTACCACCTGGTCGTGCTCGTGGACATTCT
CATGGACTTGGGGGCGCTCGCTCGAAACTGTGGACGGGAATCAAGGGTG
GATGTGGCTAAGTCAGTGACATCAGGGTTGGGATACATGCACTGCTGCC
TCAATCCGCTGCTGTATGCCTTTGTCGGGGTGAAGTTTCGAGAAAGGAT
GTGGATGCTGCTTCTGCGCCTTGGCTGTCCCAACCAAAGAGGGCTCCAA
CGACAGCCGTCAAGCAGCCGCCGCGATAGTAGCTGGAGTGAAACTAGTG
AGGCCTCCTACTCCGGACTGTGA

In certain embodiments, the exogenous CXCR3 polypeptide comprises or consists of the amino acid sequence set forth in SEQ ID NO: 28. An exemplary nucleotide sequence encoding the amino acid sequence of SEQ ID NO: 28 is set forth in SEQ ID NO: 29. SEQ ID NOS: 28 and 29 are provided below.

[SEQ ID NO: 28]
MVLEVSDHQVLNDAEVAALLENFSSSYDYGENESDSCCTSPPCPQDESL
NFDRAFLPALYSLLFLLGLLGNGAVAAVLLSRRTALSSTDTFLLHLAVA
DTLLVLTLPLWAVDAAVQWVFGSGLCKVAGALENINFYAGALLLACISF
DRYLNIVHATQLYRRGPPARVTLTCLAVWGLCLLFALPDFIFLSAHHDE
RLNATHCQYNFPQGSSSGSGCGCCSCAWAAPTREGSRGSHRLPAGIHPG
LRPQRPPTRACEAGIRAPLSPI
[SEQ ID NO: 29]
ATGGTTCTTGAAGTCAGTGATCACCAGGTACTCAACGATGCCGAAGTGG
CTGCTCTGTTGGAAAATTTTTCCTCCTCTTACGATTACGGTGAAAATGA
AAGCGATAGTTGTTGTACCAGCCCTCCCTGTCCCCAAGACTTTTCCTTG
AATTTTGATAGAGCTTTTCTGCCAGCTTTGTATTCTCTGCTGTTTCTGC
TTGGGTTGTTGGGTAACGGGGCCGTGGCCGCAGTGCTTCTTTCCCGCAG
AACCGCACTTAGCTCTACAGACACATTCCTCCTGCACTTGGCTGTAGCC
GATACTCTCCTGGTATTGACACTCCCTCTTTGGGCCGTAGATGCTGCTG
TCCAGTGGGTTTTTGGTTCCGGCCTCTGCAAAGTGGCAGGTGCTTTGTT
TAATATCAACTTTTACGCTGGGGCTTTGCTTCTCGCCTGTATTTCTTTT
GATCGATACCTCAACATTGTGCACGCTACGCAACTTTACCGCAGGGGGC
CACCAGCCCGCGTCACTCTTACCTGCCTCGCCGTATGGGGCCTCTGTCT
CCTGTTTGCGCTGCCCGACTTCATATTTTTGTCTGCACATCACGATGAA
CGATTGAACGCTACGCATTGTCAGTACAACTTTCCTCAGGGGTCCTCCA
GTGGGAGCGGGTGCGGATGCTGCTCTTGTGCATGGGCTGCACCAACAAG
AGAGGGTTCTAGAGGCTCTCATCGACTTCCCGCCGGAATCCATCCCGGA
CTGCGCCCCCAAAGGCCTCCTACCAGGGCGTGCGAAGCGGGGATTAGGG
CACCCCTTTCACCAATCTGA

In certain embodiments, the presently disclosed cell further comprises a modified promoter/enhancer at an CXCR3 gene locus, which can increase CXCR3 gene expression, e.g., a constitutive or inducible promoter placed to drive CXCR3 gene expression.

In certain embodiments, the exogenous CXCR3 polypeptide further comprises a leader or a signal peptide. The leader or a signal peptide can direct the protein (e.g., the chemokine receptor) into the secretory pathway. With the signal peptide or leader, the CXCR3 can be glycosylated and anchored in the cell membrane. The signal sequence or leader can be about 5, about 10, about 15, about 20, about 25, or about 30 amino acids in length. In certain embodiments, the leader or signal peptide comprises an IgK polypeptide. In certain embodiments, the IgK polypeptide comprises or consists of the amino acid sequence set forth in SEQ ID NO: 30. An exemplary nucleotide sequence encoding the amino acid sequence of SEQ ID NO: 30 is set forth in SEQ ID NO: 31. SEQ ID NOS: 30 and 31 are provided below.

[SEQ ID NO: 30]
METDTLLLWVLLLWVPGSTG
[SEQ ID NO: 31]
ATGGAAACTGACACACTTCTTTTGTGGGTTCTGCTCCTTTGGGTGCCTG
GAAGCACAGGA

In certain embodiments, the presently disclosed cell further comprises a soluble single-chain variable fragment (scFv) that binds a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPα, PD-L1, PD-L2, B7-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

In certain embodiments, the immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-IBB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Cells comprising a chimeric receptor and a soluble scFv that binds a polypeptide that has immunosuppressive activity or immunostimulatory activity are disclosed in International Patent Publication No. WO 2014/134165, which is incorporated by reference in its entirety.

In certain embodiments, the presently disclosed cell further comprises an exogenous CD40L. Cells comprising a chimeric receptor and an exogenous CD40L are disclosed in International Patent Publication No. WO 2014/134165.

Furthermore, in certain embodiments, the presently disclosed cell is engineered to express IL-18. In certain embodiments, the presently disclosed cell further comprises an exogenous IL-18 polypeptide or a fragment thereof. In certain embodiments, the presently disclosed cell further comprises a modified promoter/enhancer at an IL-18 gene locus, which can increase IL-18 gene expression, e.g., a constitutive or inducible promoter is placed to drive IL-18 gene expression. Cells comprising a chimeric receptor and engineered to express IL-18, e.g., comprising an exogenous IL-18 polypeptide or a fragment thereof or a modified promoter/enhancer at an IL-18 gene locus are disclosed in International Patent Publication No. WO2018/027155, which is incorporated by reference in its entirety.

Additionally or alternatively, the presently disclosed cell is engineered to express IL-33. In certain embodiments, the presently disclosed cell further comprises an exogenous IL-33 polypeptide or a fragment thereof. In certain embodiments, the presently disclosed cell further comprises a modified promoter/enhancer at an IL-33 gene locus, which can increase IL-33 gene expression, e.g., a constitutive or inducible promoter placed to drive IL-33 gene expression. Cells comprising a chimeric receptor and engineered to express IL-33, e.g., comprising an exogenous IL-33 polypeptide or a fragment thereof or a modified promoter/enhancer at an IL-33 gene locus are disclosed in International Patent Publication No. WO2019/099479, which is incorporated by reference in its entirety.

Additionally or alternatively, the presently disclosed cell is engineered to express IL-36. In certain embodiments, the presently disclosed cell further comprises an exogenous IL-36 polypeptide or a fragment thereof. In certain embodiments, the presently disclosed cell further comprises a modified promoter/enhancer at an IL-36 gene locus, which can increase IL-36 gene expression, e.g., a constitutive or inducible promoter placed to drive IL-36 gene expression. Cells comprising a chimeric receptor and engineered to express IL-36, e.g., comprising an exogenous IL-36 polypeptide or a fragment thereof or a modified promoter/enhancer at an IL-36 gene locus are disclosed in International Patent Publication No. WO2019/099483, which is incorporated by reference in its entirety.

### 5.5. Nucleic Acid Compositions and Vectors

The presently disclosed subject matter provides nucleic acid compositions comprising a polynucleotide encoding a presently disclosed chimeric receptor (e.g., one disclosed in Section 5.3).

In certain embodiments, the nucleic acid composition further comprises a promoter that is operably linked to the polynucleotide.

In certain embodiments, the promoter is endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and gamma retrovirus 5′ long-terminal repeat (LTR) promoter. In certain embodiments, the promoter is an inducible promoter. In certain embodiment, the inducible promoter is selected from the group consisting of a NFAT transcriptional response element (TRE) promoter, a CD69 promoter, a CD25 promoter, Nur77 promoter, and an IL-2 promoter.

Also provided are vectors comprising such nucleic acid compositions, and cells comprising such nucleic acid compositions or vectors.

In certain embodiments, the nucleic acid composition further comprises a cleavable (e.g., self-cleavable) linker. In certain embodiments, the cleavable linker is a 2A peptide, e.g., a P2A peptide, a T2A peptide, an E2A peptide, and a F2A peptide. In certain embodiments, the cleavable linker is a T2A peptide. In certain embodiments, the cleavable linker is a P2A peptide. In certain embodiments, the P2A peptide comprises or consists of the amino acid sequence set forth in SEQ ID NO: 32. An exemplary nucleotide sequence encoding the amino acid sequence of SEQ ID NO: 32 is set forth in SEQ ID NO: 33. SEQ ID NOS: 32 and 33 are provided below.

[SEQ ID NO: 32]
GSGATNFSLLKQAGDVEENPGP
[SEQ ID NO: 33]
GGATCTGGAGCAACAAACTTCTCACTACTCAAACAAGCAGGTGACGTGG
AGGAGAATCCCGGACCC

The compositions and nucleic acid compositions can be administered to subjects or and/delivered into cells by art-known methods or as described herein.

Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding chimeric receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed chimeric receptor, a retroviral vector can be employed for transduction, however any other suitable viral vector or non-viral delivery system can be used. The chimeric receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed chimeric receptor to a cell or a subject. In certain embodiments, a CRISPR system is used to deliver a presently disclosed chimeric receptor. In certain embodiments, zinc-finger nucleases are used to deliver the chimeric receptor. In certain embodiments, a TALEN system is used to deliver a presently disclosed chimeric receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying chimeric receptor expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying chimeric receptor expression cassette, ZFNs can be used to insert the chimeric receptor expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### 5.5.1. Methods of Delivering

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642, which are incorporated by reference in their entireties.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-diolcoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioetylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z, 12″Z, 12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), diolcoylphosphatidylcholine (DOPC), dipalmitoylphosphatidylcholine (DPPC), diolcoylphosphatidylglycerol (DOPG), dipalmitoylphosphatidylglycerol (DPPG), diolcoylphosphatidylethanolamine (DOPE), palmitoylolcoylphosphatidylcholine (POPC), palmitoylolcoyl-phosphatidylethanolamine (POPE) and diolcoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 5.6. Polypeptides and Analogs

The presently disclosed subject matter provides methods for optimizing an amino acid sequence or a nucleotide sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, mesothelin, CD8, CD28, CD3ζ, etc.). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least about 5, about 10, about 15 or about 20 amino acid residues, e.g., at least about 25, about 50, or about 75 amino acid residues, or more than about 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least about 5, about 10, about 13, or about 15 amino acids. In certain embodiments, a fragment comprises at least about 20 contiguous amino acids, at least about 30 contiguous amino acids, or at least about 50 contiguous amino acids. In certain embodiments, a fragment comprises at least about 60 to about 80, about 100, about 200, about 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5.7. Formulations and Administration

The presently disclosed subject matter also provides compositions comprising the presently disclosed cells. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for inducing and/or enhancing an immune response to an antigen and/or treating and/or preventing a tumor, e.g., a tumor associated with MUC16. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, intravesicular administration, intracranial administration, intraocular administration, subcutaneous administration, intranodal administration, intratumoral administration, intrathecal administration, intrapleural administration, intraosseous administration, intraperitoneal administration, pleural administration, and direct administration to the subject.

Additionally or alternatively, the presently disclosed subject matter also provides compositions comprising lipid nanoparticles (e.g., described in Section 5.5.1) including a nucleic acid or a nucleic acid composition disclosed herein. Compositions comprising the presently disclosed lipid nanoparticles can be conveniently provided as sterile and/or pyrogen-free. Compositions can be prepared to meet the standards of the United States Pharmacopoeia (USP), the European Pharmacopoeia (EP), the British Pharmacopoeia, and/or the International Pharmacopoeia.

Compositions including the presently disclosed lipid nanoparticles can include pharmaceutically acceptable excipients. Non-limiting examples of pharmaceutically acceptable excipients include inert diluents, dispersing agents, granulating agents, surface active agents, emulsifiers, disintegrating agents, binding agents, preservatives, buffering agents, lubricating agents, and/or oils. Furthermore, excipients such as cocoa butter and suppository waxes, coloring agents, coating agents, sweetening, flavoring, and/or perfuming agents can be present in the composition.

In certain embodiments, compositions including the presently disclosed lipid nanoparticles can be prepared as injectable preparations. These injectable preparations can include pharmaceutically acceptable vehicles and solvents including, without any limitation, water, Ringer's solution, U.S.P., isotonic sodium chloride solution, and/or oils (e.g., oleic acid). In certain embodiments, injectable preparations comprising the presently disclosed lipid nanoparticles can include a liquid suspension of crystalline or amorphous material with poor water solubility. Use of these poor water solubility materials allows to slow absorption from subcutaneous or intramuscular injection. Alternatively or additionally, compositions including the presently disclosed lipid nanoparticles can be prepared for rectal or vaginal administration, oral administration, topical and/or transdermal administration, intradermal administration, pulmonary administration, nasal administration, buccal administration, or ophthalmic administration. Additional information on various ways for formulating and preparing pharmaceutical compositions including the presently disclosed lipid nanoparticles can be found in Remington: The Science and Practice of Pharmacy, 22nd Edition, A. R. Gennaro, Lippincott, Williams & Wilkins, Baltimore, Md., 2012.

In certain embodiments, the compositions including the presently disclosed lipid nanoparticles can be formulated for controlled release or sustained release. As used herein, the term “controlled release” refers to a pharmaceutical composition or compound release profile that conforms to a particular pattern of release to effect a therapeutic outcome. As used herein, the term “sustained release” refers to a pharmaceutical composition or compound that conforms to a release rate over a specific period of time. The period of time may include, but is not limited to, hours, days, weeks, months and years.

Compositions comprising the presently disclosed lipid nanoparticles can be provided systemically or directly to a subject for inducing and/or enhancing an immune response to an antigen and/or treating and/or preventing a tumor, e.g., a tumor associated with MUC16. In certain embodiments, the presently disclosed lipid nanoparticles or compositions comprising thereof are provided in vivo to immunoresponsive cells. In certain embodiments, the presently disclosed lipid nanoparticles or compositions comprising thereof are directly injected into an organ of interest (e.g., an organ affected by a neoplasia). Alternatively, the presently disclosed lipid nanoparticles or compositions comprising thereof are provided indirectly to the organ of interest, for example, by administration into the circulatory system (e.g., the tumor vasculature). In certain embodiments, the presently disclosed lipid nanoparticles or compositions comprising thereof are provided ex vivo to immunoresponsive cells. Expansion and differentiation agents can be provided prior to, during or after administration of the lipid nanoparticles or compositions to increase production of cells (e.g., T cells or NK cells) ex vivo or in vivo.

The presently disclosed lipid nanoparticles can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of the presently disclosed lipid nanoparticles are administered to a subject. In certain embodiments, between about 0.005 mg/kg to about 2.5 mg/kg, from about 0.1 mg/kg to about 1 mg/kg, or from about 0.05 mg/kg to about 1 mg/kg of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art. Dosages can be readily adjusted by those skilled in the art (e.g., a decrease in purity may require an increase in dosage).

### 5.8. Methods of Treatment

The presently disclosed cells and compositions comprising thereof can be used in a therapy or medicament. The presently disclosed subject matter provides various methods of using the cells (e.g., T cells) or compositions comprising thereof. For example, the presently disclosed cells and compositions comprising thereof can be used for reducing tumor burden in a subject. The presently disclosed cell can reduce the number of tumor cells, reduce tumor size, and/or eradicate the tumor in the subject. The presently disclosed cells and compositions comprising thereof can be used for treating and/or preventing a disease or disorder associated with MUC16 in a subject. The presently disclosed cells and compositions comprising thereof can be used for treating and/or preventing a tumor in a subject. The presently disclosed cells and compositions comprising thereof can be used for prolonging the survival of a subject suffering from a disease or disorder associated with MUC16. The presently disclosed cells and compositions comprising thereof can be used for prolonging the survival of a subject suffering from a disease or disorder associated with MUC16 or a tumor. Such methods comprise administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter provides methods of reducing tumor burden in a subject. In certain embodiments, the method of reducing tumor burden comprises administering to the subject the presently disclosed cells or a composition comprising thereof. The presently disclosed cell can reduce the number of tumor cells, reduce tumor size, and/or eradicate the tumor in the subject.

The presently disclosed subject matter also provides methods of increasing or lengthening survival of a subject having a tumor or a disease or disorder associated with MUC16. In certain embodiments, the method of increasing or lengthening survival of a subject having a tumor or a disease or disorder associated with MUC16 comprises administering to the subject the presently disclosed cells or a composition comprising thereof. The method can reduce or eradicate tumor burden in the subject.

Additionally, the presently disclosed subject matter provides methods for increasing an immune response in a subject, comprising administering to the subject the presently disclosed cells or a composition comprising thereof.

The presently disclosed subject matter further provides methods for treating and/or preventing a tumor or a disease or disorder associated with MUC16 in a subject. In certain embodiments, the method of treating and/or preventing a tumor or a disease or disorder associated with MUC16 comprises administering to the subject the presently disclosed cells or a composition comprising thereof.

In certain embodiments, the disease or disorder associated with MUC16 is a tumor associated with MUC16. In certain embodiments, the tumor is associated with MUC16. In certain embodiments, the tumor is associated with overexpression of MUC16. In certain embodiments, the tumor is cancer. In certain embodiments, the tumor is selected from the group consisting of ovarian cancer, pancreatic cancer, bladder cancer, lung cancer, stomach cancer, endometrial cancer, breast cancer, colorectal cancer, thyroid cancer, and head and neck squamous cell cancers. In certain embodiments, the tumor is selected from the group consisting of ovarian cancer, pancreatic cancer, bladder cancer, lung cancer, and stomach cancer.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of a presently disclosed chimeric receptor, adoptively transferred cells (e.g., immunoresponsive cells, e.g., T cells or NK cells) are endowed with augmented and selective cytolytic activity at the tumor site. Furthermore, subsequent to their localization to tumor and their proliferation, the cells turn the tumor into a highly conductive environment for a wide range of immune cells involved in the physiological anti-tumor response (tumor infiltrating lymphocytes, NK-, NKT-cells, dendritic cells, and macrophages).

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the chimer receptor. The suicide gene can be included within the vector comprising nucleic acids encoding a presently disclosed chimeric receptor. In this way, administration of a prodrug designed to activate the suicide gene (e.g., a prodrug (e.g., AP1903 that can activate iCasp-9) during malignant T-cell transformation or T cell-mediated toxicity (e.g., GVHD) triggers apoptosis in the suicide gene-activated cells expressing the chimeric receptor. The incorporation of a suicide gene into the a presently disclosed chimeric receptor gives an added level of safety with the ability to eliminate the majority of receptor-expressing cells within a very short time period. A presently disclosed cell (e.g., a T cell) incorporated with a suicide gene can be pre-emptively eliminated at a given timepoint post the cell infusion, or eradicated at the earliest signs of toxicity.

### 5.9. Kits

The presently disclosed subject matter provides kits for inducing and/or enhancing an immune response in a subject, treating and/or preventing a disease or disorder associated with MUC16 or a tumor in a subject, reducing tumor burden in a subject, and/or increasing or lengthening survival of a subject having a tumor or a disease or disorder associated with MUC16 in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit includes a nucleic acid molecule encoding a presently disclosed chimeric receptor.

If desired, the cells and/or nucleic acid molecules are provided together with instructions for administering the cells or nucleic acid molecules to a subject having or at risk of developing a tumor. The instructions generally include information about the use of the composition for the treatment and/or prevention of a tumor. In certain embodiments, the instructions include at least one of the following: description of the therapeutic agent; dosage schedule and administration for treatment or prevention of a tumor; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides a chimeric receptor, comprising an extracellular domain, a transmembrane domain, and an intracellular signaling domain, wherein the extracellular domain comprises a mesothelin polypeptide that binds to MUC16.
A2. The foregoing chimeric receptor of A1, wherein the mesothelin polypeptide comprises or consists of an amino acid sequence that is at least about 80% identical to the amino acid sequence set forth in SEQ ID NO: 1 or a fragment thereof.
A3. The foregoing chimeric receptor of A1 or A2, wherein the mesothelin polypeptide comprises or consists of an amino acid sequence that is at least about 80% identical to amino acids 296 to 598 of SEQ ID NO: 1 or a fragment thereof.
A4. The foregoing chimeric receptor of any one of A1-A3, wherein the mesothelin polypeptide comprises or consists of amino acids 296 to 598 of SEQ ID NO: 1.
A5. The foregoing chimeric receptor of A1, wherein the mesothelin polypeptide comprises an amino acid sequence that is within Region I of human mesothelin.
A6. The foregoing chimeric receptor of A1 or A6, wherein the mesothelin polypeptide comprises an amino acid sequence that is within Region IAB of human mesothelin.
A7. The foregoing chimeric receptor of A5 or A6, wherein the human mesothelin comprises or consists of the amino acid sequence set forth in SEQ ID NO: 1.
A8. The foregoing chimeric receptor of A7, wherein the Region I comprises or consists of amino acids 296 to 390 of SEQ ID NO: 1.
A9. The foregoing chimeric receptor of A7, wherein the Region IAB comprises or consists of amino acids 296 to 359 of SEQ ID NO: 1.
A10. The foregoing chimeric receptor of any one of A5-A9, wherein the mesothelin polypeptide comprises or consists of an amino acid sequence that is at least about 80% identical to the amino acid sequence of amino acids 296 to 359 of SEQ ID NO: 1 or a fragment thereof.
A11. The foregoing chimeric receptor of any one of A5-A10, wherein the mesothelin polypeptide comprises or consists of amino acids 296 to 359 of SEQ ID NO: 1.
A12. The foregoing chimeric receptor of any one of A1-A11, wherein a signal peptide is covalently joined to the N-terminus of the extracellular domain.
A13. The foregoing chimeric receptor of A12, wherein the signal peptide comprises or consists of a CD8 polypeptide.
A14. The foregoing chimeric receptor of A13, wherein the CD8 polypeptide comprises or consists of the amino acid sequence set forth in SEQ ID NO: 4.
A15. The foregoing chimeric receptor of any one of A1-A14, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM polypeptide, a CTLA-4 polypeptide, a CD27 polypeptide, a CD40 polypeptide, a NKGD polypeptide, a PD-1 polypeptide, a LAG-3 polypeptide, a 2B4 polypeptide, a BTLA polypeptide, or a combination thereof.
A16. The foregoing chimeric receptor of A15, wherein the transmembrane domain comprises a CD28 polypeptide.
A17. The foregoing chimeric receptor of A16, wherein the CD28 polypeptide comprises or consists of amino acids 153 to 179 of SEQ ID NO: 7.
A18. The foregoing chimeric receptor of any one of A1-A17, further comprising a hinge/spacer region.
A19. The foregoing chimeric receptor of A18, wherein the hinge/spacer region comprises a CD28 polypeptide.
A20. The foregoing chimeric receptor of A19, wherein the CD28 polypeptide comprises or consists of amino acids 114 to 152 of SEQ ID NO: 7.
A21. The foregoing chimeric receptor of any one of A1-A20, wherein the intracellular signaling domain comprises a CD3ζ polypeptide.
A22. The foregoing chimeric receptor of A21, wherein the CD3ζ polypeptide comprises or consists of the amino acid sequence set forth in SEQ ID NO: 14.
A23. The foregoing chimeric receptor of any one of A1-A22, wherein the intracellular signaling domain further comprises at least one co-stimulatory signaling region.
A24. The foregoing chimeric receptor of A23, wherein the at least one co-stimulatory signaling region comprises a CD28 polypeptide, a 4-1BB polypeptide, an OX40 polypeptide, an ICOS polypeptide, a DAP-10 polypeptide, or a combination thereof.
A25. The foregoing chimeric receptor of A24, wherein the at least one co-stimulatory signaling region comprises a CD28 polypeptide.
A26. The foregoing chimeric receptor of A25, wherein the CD28 polypeptide comprises or consists of amino acids 180 to 220 of SEQ ID NO: 7.
A27. The foregoing chimeric receptor of A25, wherein the CD28 polypeptide comprises a mutated YMNM motif.
A28. The foregoing chimeric receptor of A25 or A27, wherein the CD28 polypeptide comprises or consists of the amino acid sequence set forth in SEQ ID NO: 85, SEQ ID NO: 86, SEQ ID NO: 87, SEQ ID NO: 88, SEQ ID NO: 89.
A29. The foregoing chimeric receptor of any one of A1-A28, wherein the chimeric receptor is recombinantly expressed or expressed from a vector.
B1. In certain non-limiting embodiments, the presently disclosed subject matter provides a cell comprising the chimeric receptor of any one of A1-A29.
B2. The foregoing cell of B1, wherein the chimeric receptor is constitutively expressed on the surface of the cell.
B3. The foregoing cell of B1 or B2, wherein the cell overexpresses a chemokine receptor.
B4. The foregoing cell of B1 or B2, wherein the cell comprises an exogenous chemokine receptor or a fragment thereof.
B5. The foregoing cell of B3 or B4, wherein the chemokine receptor is CXCR3.
B6. The foregoing cell of any one of B3-B5, wherein the chemokine receptor is CXCR3A.
B7. The foregoing cell of B1 or B2, wherein the cell comprises an exogenous Il-18 polypeptide or a fragment thereof.
B8. The foregoing cell of B1 or B2, wherein the cell comprises a modified promoter/enhancer at an IL-18 gene locus to thereby increase IL-18 gene expression.
B9. The foregoing cell of any one of B1-B8, wherein the cell is an immunoresponsive cell.
B10. The foregoing cell of any one of B1-B9, wherein the cell is a cell of the lymphoid lineage or a cell of the myeloid lineage.
B11. The foregoing cell of any one of B1-B10, wherein the cell is selected from the group consisting of a T cell, a Natural Killer (NK) cell, a B cell, a monocyte, and a macrophage, a stem cell from which a lymphoid cell may be differentiated, a stem cell from which a myeloid cell may be differentiated, and combinations thereof.
B12. The foregoing cell of B11, wherein the cell is a T cell.
B13. The foregoing cell of B12, wherein the T cell is selected from the group consisting of a helper T cell, a cytotoxic T cell, a memory T cell, an effector memory T cell, a regulatory T cell, a tumor-infiltrating lymphocyte (TIL), a natural killer T cell, a mucosal associated invariant T cell, a γδ T cell, and combinations thereof.
B14. The foregoing cell of B12 or B13, wherein the T cell is a cytotoxic T cell.
B15. The foregoing cell of B13, wherein the stem cell is a pluripotent stem cell.
B16. The foregoing cell of B15, wherein the pluripotent stem cell is an embryoid stem cell or an induced pluripotent stem cell.
C1. In certain non-limiting embodiments, the presently disclosed subject matter provides a nucleic acid molecule encoding the chimeric receptor of any one of A1-A29.
C2. In certain non-limiting embodiments, the presently disclosed subject matter provides a nucleic acid composition comprising a first polynucleotide encoding the chimeric receptor of any one of A1-A29 and a second polynucleotide encoding a chemokine receptor.
C3. The foregoing nucleic acid composition of C2, wherein the chemokine receptor is CXCR3.
C4. In certain non-limiting embodiments, the presently disclosed subject matter provides a nucleic acid composition comprising a first polynucleotide encoding the chimeric receptor of any one of A1-A29 and a second polynucleotide encoding an IL-18 polypeptide.
D1. In certain non-limiting embodiments, the presently disclosed subject matter provides a vector comprising the nucleic acid molecule of C1 or the nucleic acid composition of any one of C2-C4.
D2. The foregoing vector of D1, wherein the vector is a retroviral vector.
D3. The foregoing vector of D2, wherein the retroviral vector is a γ-retroviral vector or a lentiviral vector.
E1. In certain non-limiting embodiments, the presently disclosed subject matter provides a lipid nanoparticle comprising the nucleic acid molecule of C1 or the nucleic acid composition of any one of C2-C4.
F1. In certain non-limiting embodiments, the presently disclosed subject matter provides a host cell expressing the nucleic acid molecule of C1 or the nucleic acid composition of any one of C2-C4.
F2. The foregoing host cell of F1, wherein the host cell is a T cell.
G1. In certain non-limiting embodiments, the presently disclosed subject matter provides a composition comprising the cell of any one of B1-B16.
G2. The foregoing composition of G1, which is a pharmaceutical composition further comprising a pharmaceutically acceptable carrier.
G3. In certain non-limiting embodiments, the presently disclosed subject matter provides a composition comprising the lipid nanoparticle of E1.
G4. The foregoing composition of G3, which is a pharmaceutical composition further comprising a pharmaceutically acceptable carrier.
H1. In certain non-limiting embodiments, the presently disclosed subject matter provides a method of reducing tumor burden in a subject having a tumor, comprising administering to the subject the cell of any one of B1-B16 or the composition of any one of G1-G4.
H2. The foregoing method of H1, wherein the method reduces the number of tumor cells, reduces tumor size, and/or eradicates the tumor in the subject.
H3. In certain non-limiting embodiments, the presently disclosed subject matter provides a method of increasing or lengthening survival of a subject having a tumor, comprising administering to the subject the cell of any one of B1-B16 or the composition of any one of G1-G4.
H4. In certain non-limiting embodiments, the presently disclosed subject matter provides a method of treating and/or preventing a tumor in a subject, comprising administering to the subject the cell of any one of B1-B16 or the composition of any one of G1-G4.
H5. The foregoing method of any one of H1-H4, wherein the tumor is associated with MUC16.
H6. The foregoing method of any one of H1-H5, wherein the tumor is a cancer.
H7. The foregoing method of any one of H1-H6, wherein the tumor is selected from the group consisting of ovarian cancer, pancreatic cancer, bladder cancer, lung cancer, stomach cancer, endometrial cancer, breast cancer, colorectal cancer, thyroid cancer, and head and neck squamous cell cancers.
H8. The foregoing method of any one of H1-H7, wherein the tumor is selected from the group consisting of ovarian cancer, pancreatic cancer, bladder cancer, lung cancer, and stomach cancer.
H9. The foregoing method of any one of H1-H8, wherein the tumor is bladder cancer.
H10. The foregoing cell of any one of B1-B18 or the foregoing composition of any one of G1-G4 for use in reducing tumor burden in a subject having a tumor, increasing or lengthening survival of a subject having a tumor, treating a tumor in a subject, or preventing a tumor in a subject.
H11. The foregoing cell or the foregoing composition for use of H10, wherein the tumor is associated with MUC16.
H12. The foregoing cell or the foregoing composition for use of H10 or H11, wherein the tumor is a cancer.
H13. The foregoing cell or the foregoing composition for use of any one of H10-H12, wherein the tumor is selected from the group consisting of ovarian cancer, pancreatic cancer, bladder cancer, lung cancer, stomach cancer, endometrial cancer, breast cancer, colorectal cancer, thyroid cancer, and head and neck squamous cell cancers.
H14. The foregoing cell or the foregoing composition for use of any one of H10-H13, wherein the tumor is selected from the group consisting of ovarian cancer, pancreatic cancer, bladder cancer, lung cancer, and stomach cancer.
I1. In certain non-limiting embodiments, the presently disclosed subject matter provides a method for producing a cell comprising a chimeric receptor of any one of A1-A29, comprising introducing into the cell a nucleic acid molecule that encodes the chimeric receptor.
J1. In certain non-limiting embodiments, the presently disclosed subject matter provides a kit for reducing tumor burden in a subject, treating and/or preventing a tumor in a subject, and/or increasing or lengthening survival of a subject having a tumor, comprising the cell of any one of B1-B16.
J2. The foregoing kit of J1, wherein the kit further comprises written instructions for using the cell for reducing tumor burden in a subject, treating and/or preventing a tumor or neoplasm in a subject, and/or increasing or lengthening survival of a subject having a tumor.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—Identification of MUC16 as Target

MUC16/CA125 is a glycoprotein with limited expression in normal tissues, which is expressed on multiple cancer cell types (including ovarian, pancreatic, bladder, and lung and stomach among others), and functions to promote metastatic invasion, immune evasion, motility, and chemotherapeutic resistance. A novel chimeric receptor composed of human mesothelin was generated. This chimeric receptor endows immune cells to become activated upon binding MUC16 (CA125) and initiate anti-tumor responses. In addition, a separate chimeric receptor was generated, wherein the separate chimeric receptor is composed of a smaller segment of human mesothelin, representing the dominant binding domain that binds to MUC16, fused to the same intracellular domains for the same purpose. In addition, a separate mouse chimeric receptor composed of mouse mesothelin with mouse intracellular domains that binds to MUC16 was generated. This is a ligand-based chimeric receptor targeting MUC16 and is ideal for oncologic applications. Mesothelin has a high binding affinity for MUC16, is human in origin therefore is less immunogenic than antibody scFv-based MUC16 chimeric antigen receptors (e.g., a chimeric antigen receptor (CAR) including an scFv) and can compete with MUC16 interacting with host's native mesothelin.

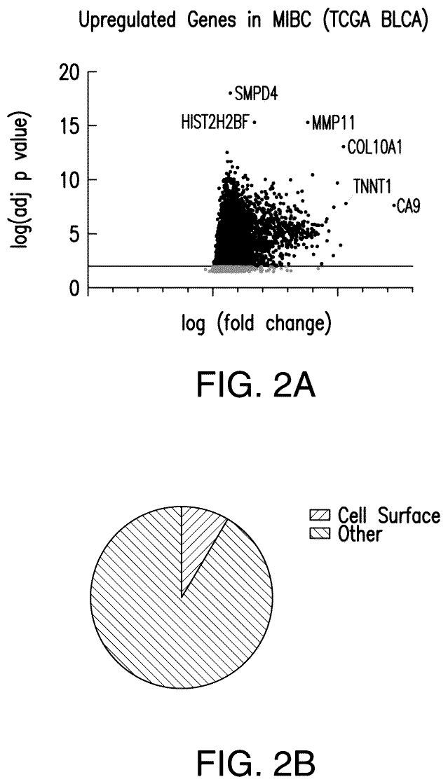

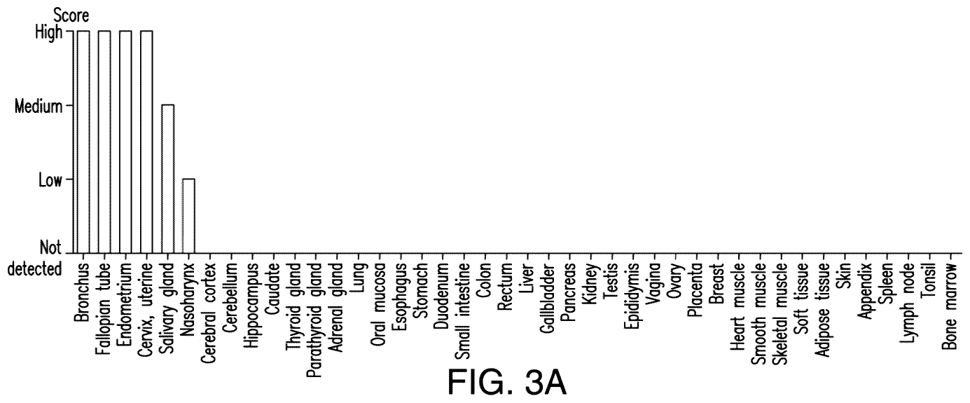

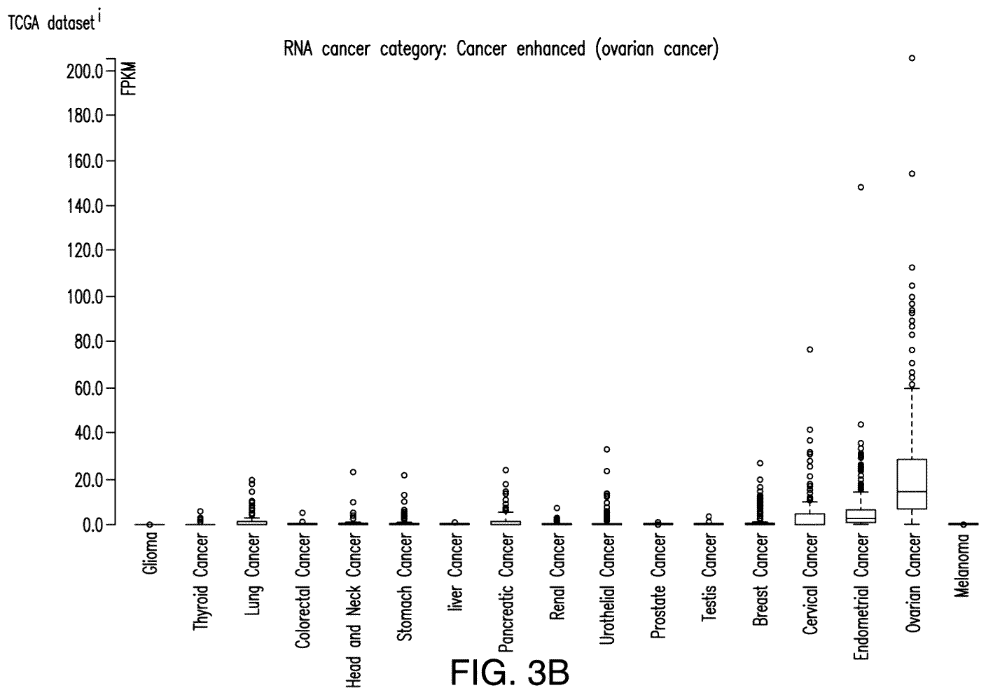

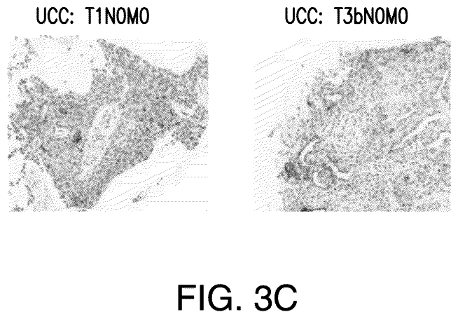

In silico analysis was initially performed to identify target molecules for bladder cancer. Analysis were conducted by integrating data from The Cancer Genome Atlas (TCGA), UniProt, the Genotype-Tissue Expression (GTEx), and the Cancer Cell Line Encyclopedia (CCLE) in a unique pipeline. More than 1825 genes were upregulated in muscle-invasive bladder cancer as compared to normal cells (see FIG. 2A). Of these, about 155 gene were found to encode for a membrane-bound proteins (see FIG. 2B) and up to 48 genes were found to be upregulated in bladder cancer with minimal expression in normal tissues. As shown in FIGS. 3A-3C, MUC16 was identified as an optimal target for bladder cancer.

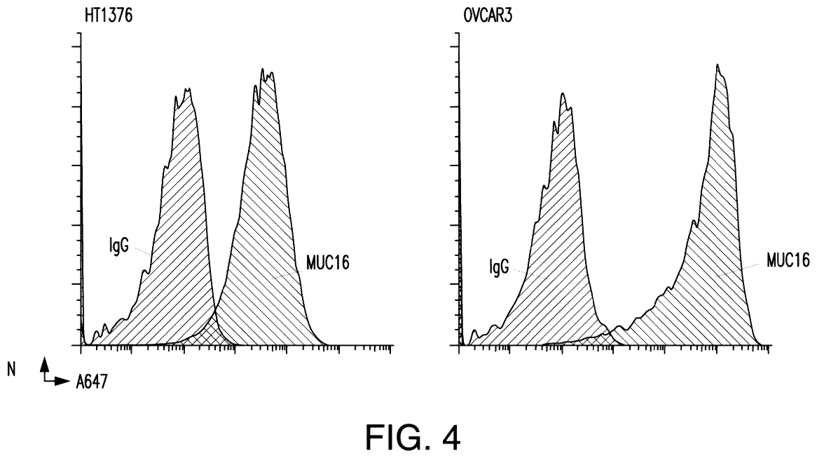

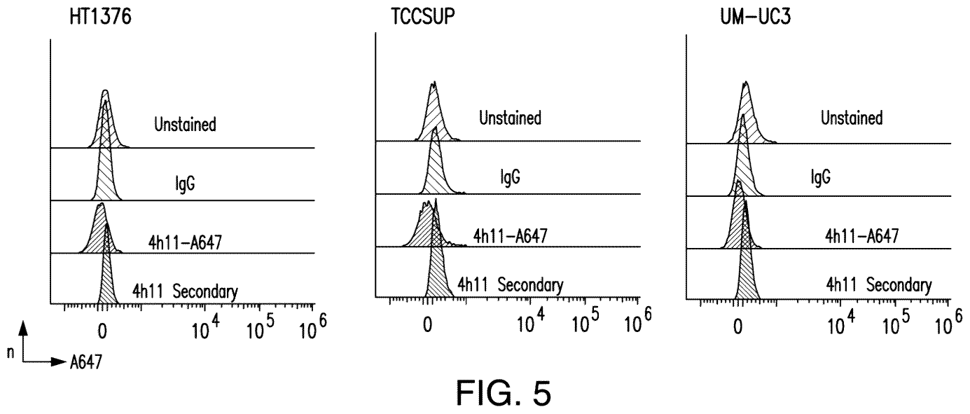

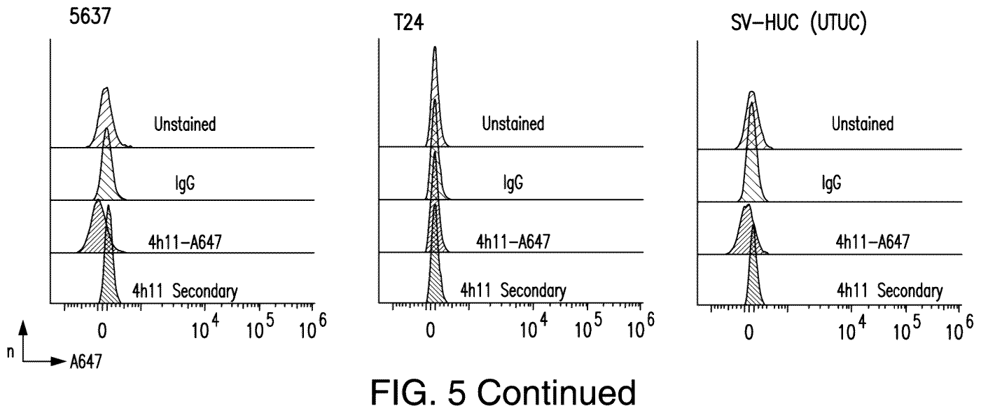

As shown in FIGS. 4 and 5, MUC16 was expressed in bladder cancer (HT1376) and ovarian cancer (OVCAR3) cell lines. Two anti-MUC16 antibodies 3A5 and 4H11 were also tested. The 3A5 antibody recognizes the tandem repeats of MUC16. The 4H11 antibody recognizes the C-terminus of MUC16. Surprisingly, antibody 4H11 did not bind to common bladder cancer cell lines, e.g., HT1376. These data demonstrate the need for an alternative way to target MUC16.

### Example 2—T Cells Comprising the Presently Disclosed Chimeric Receptor and their Antitumor Activities

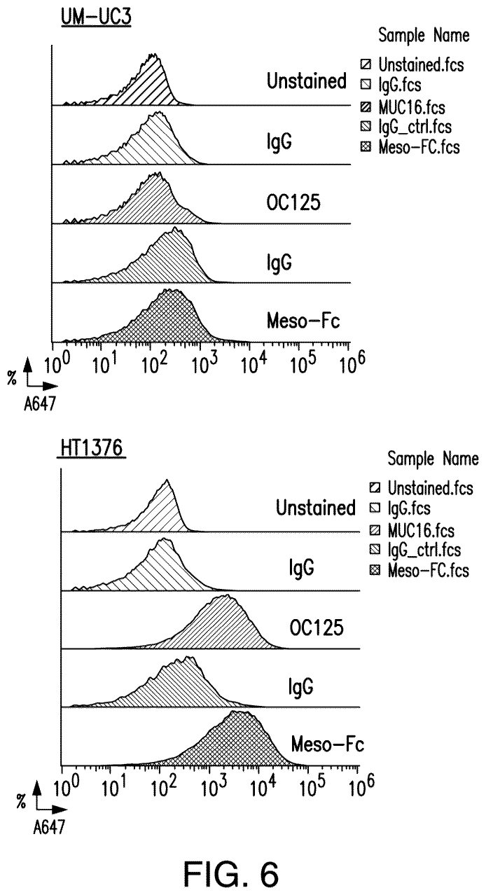

To test the hypothesis that mesothelin can target MUC16, a mesothelin-Fc protein was incubated with cancer bladder cells expressing MUC16 (HT1376) or negative control (UM-UC3). As shown in FIG. 6, mesothelin-Fc was able to bind to MUC16+ cells but not to the negative control.

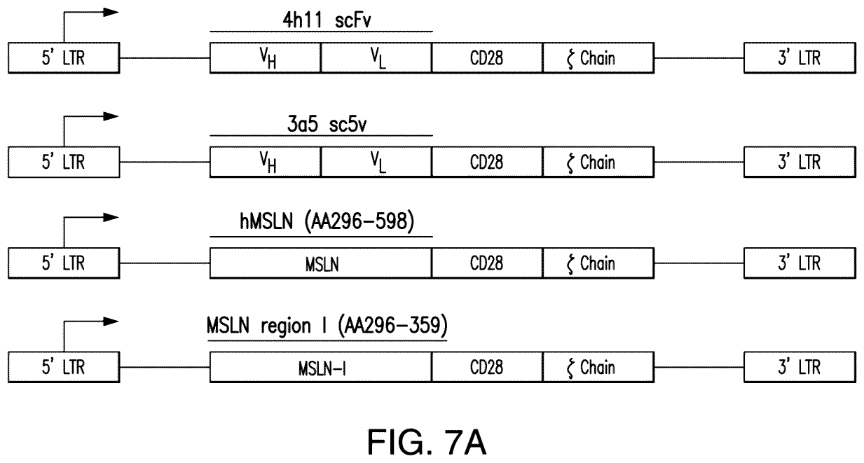

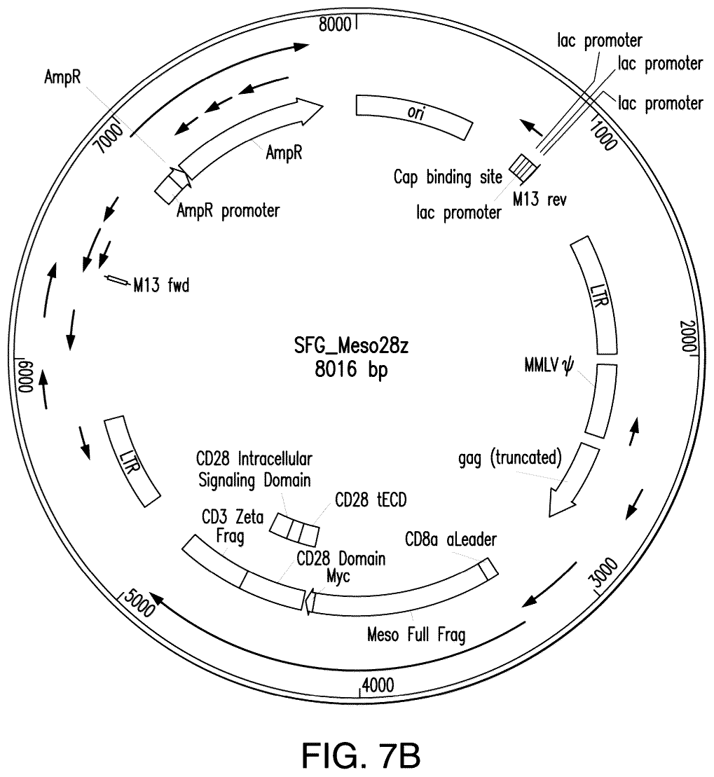

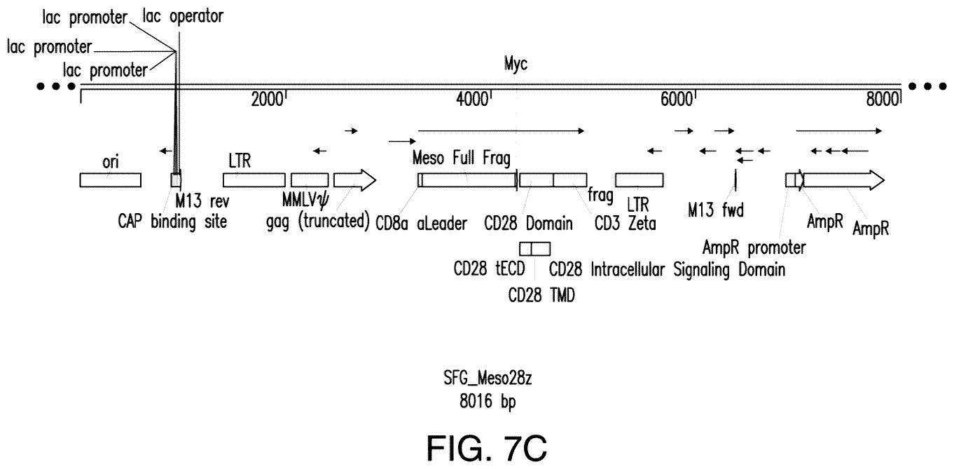

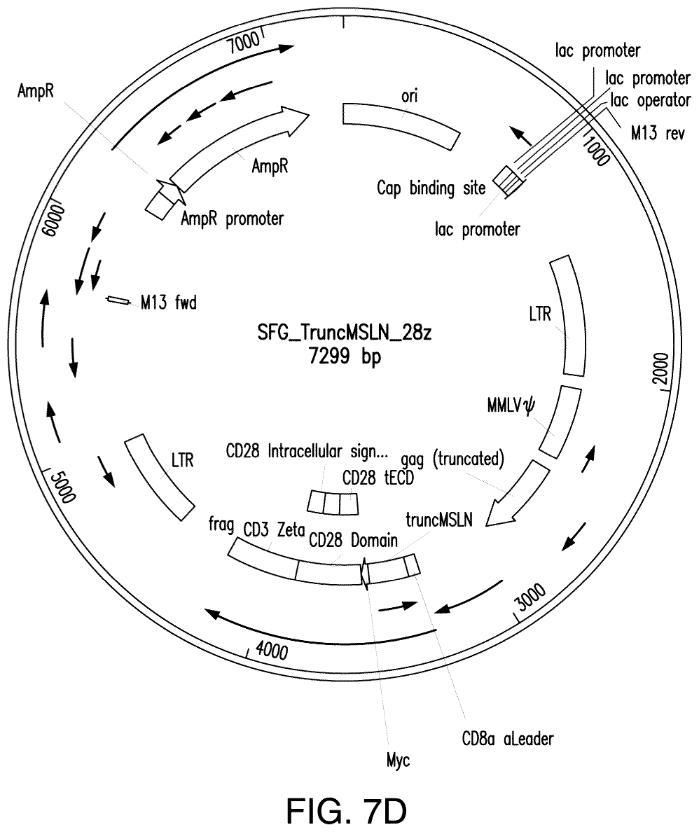

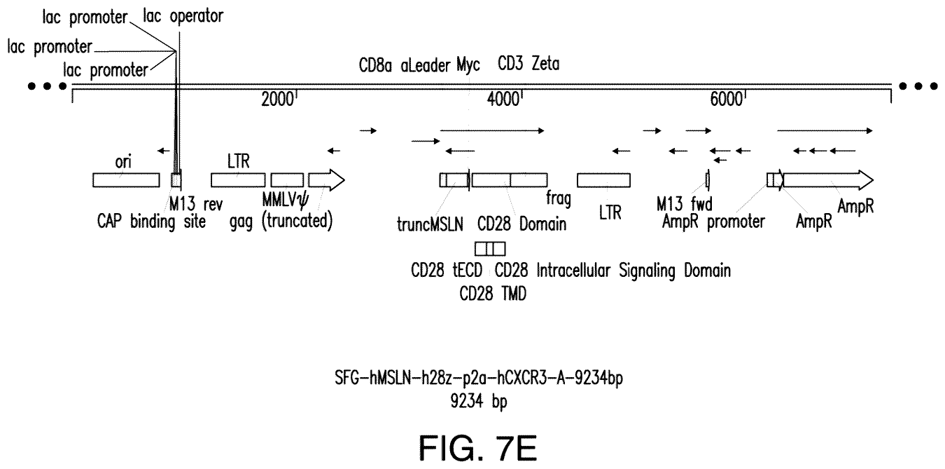

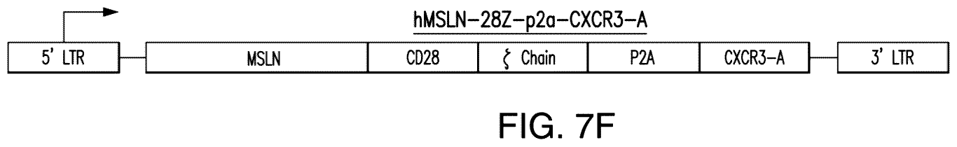

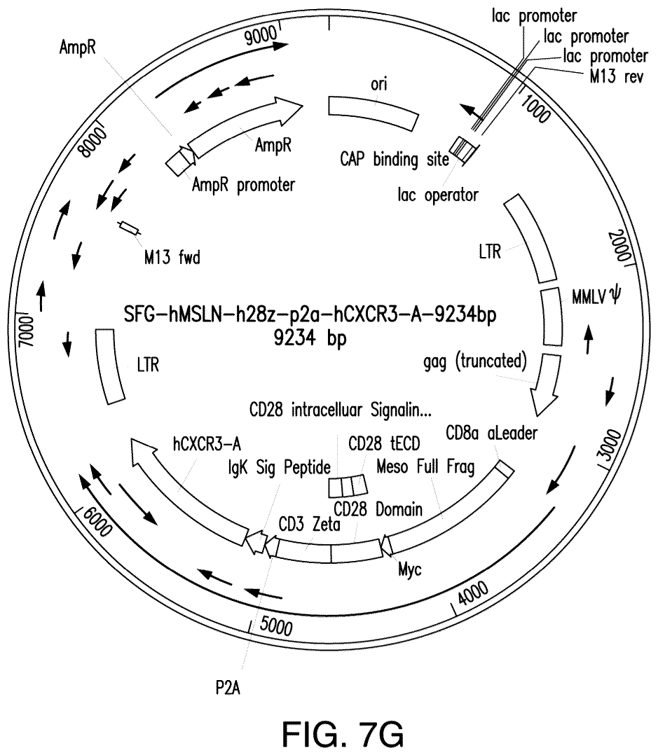

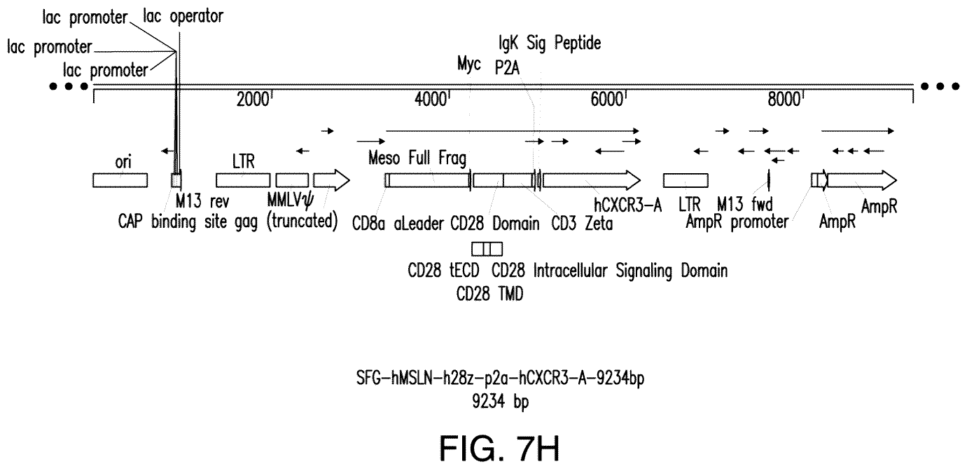

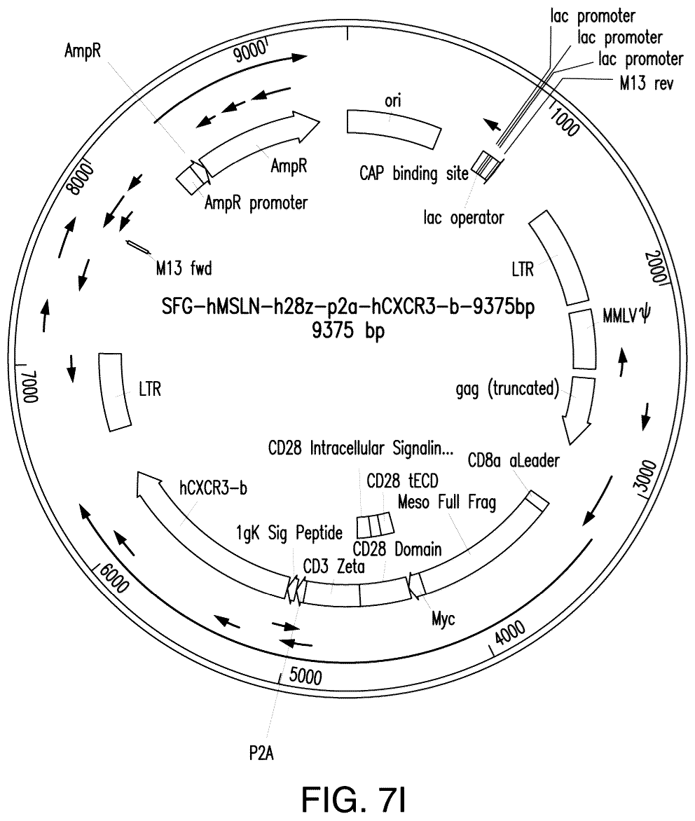

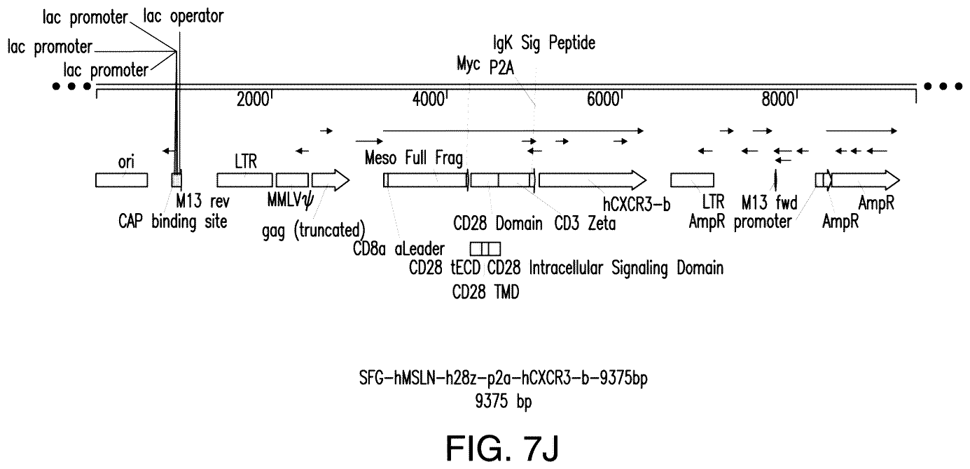

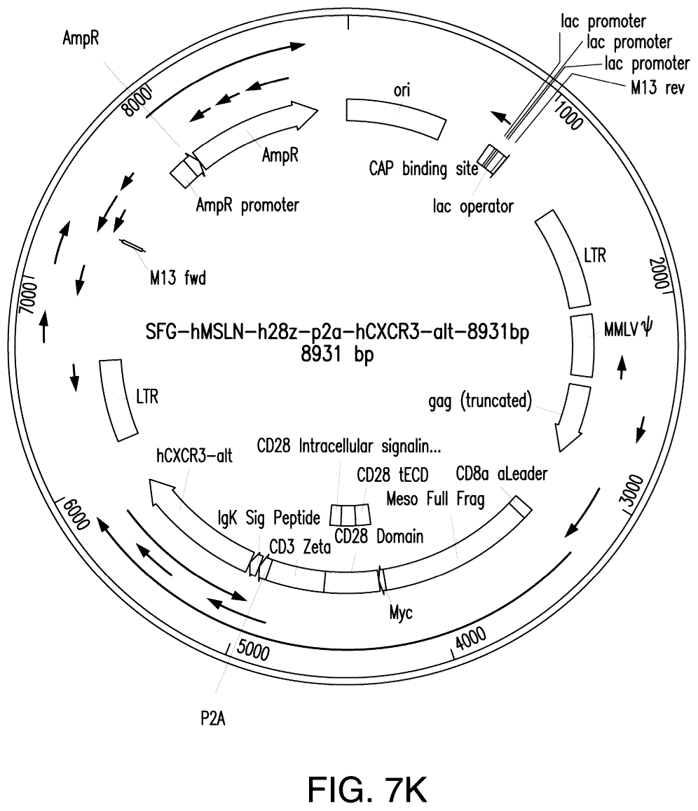

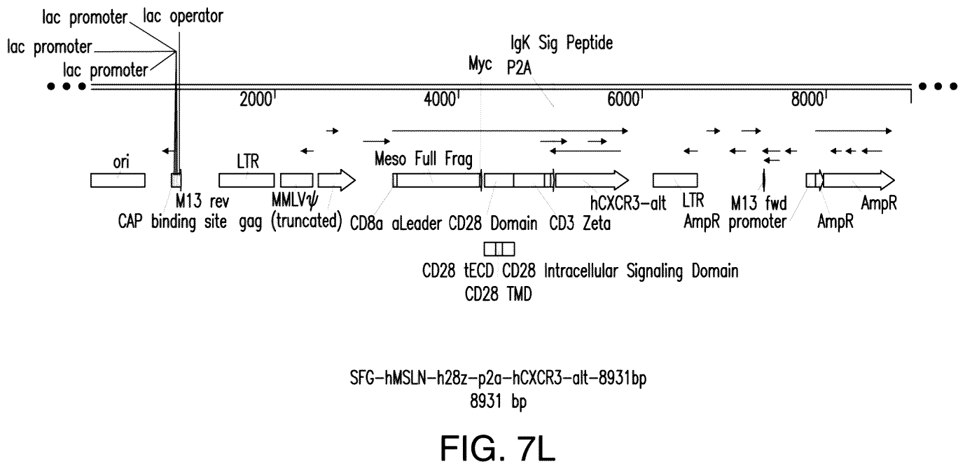

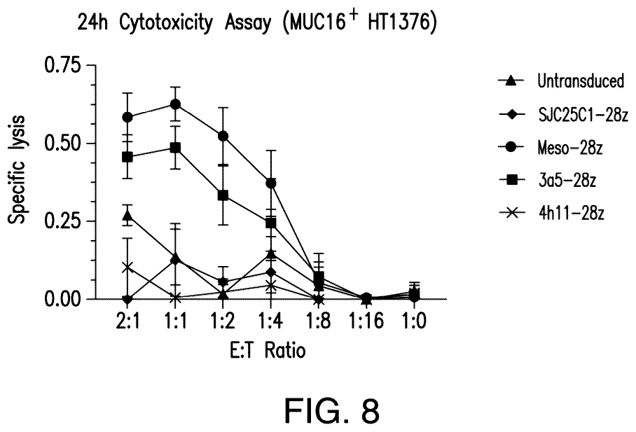

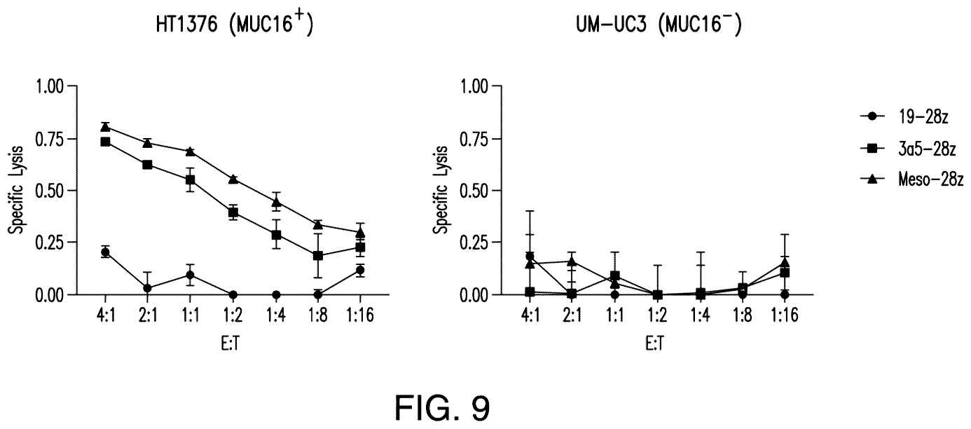

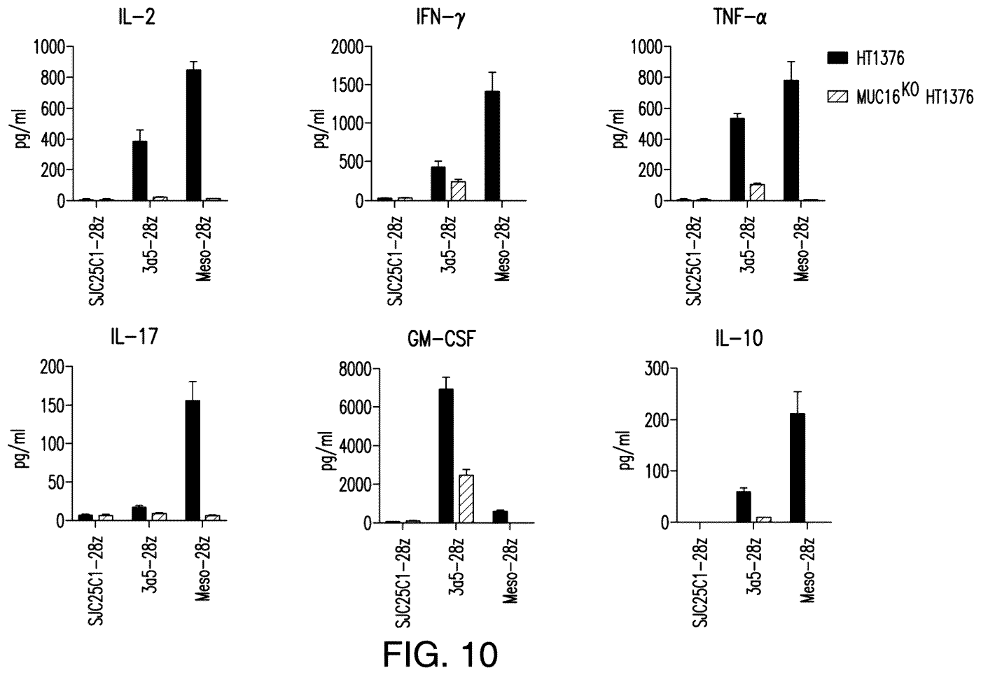

Thus, T cells comprising presently disclosed chimeric receptors comprising an extracellular domain that comprises a mesothelin polypeptide were developed, e.g., Meso-28z and TruncMSLN_28 z (see FIGS. 7A-7E). Additionally, a chimeric antigen receptor (CAR) including the scFv of antibody 4H11 (“4h11-28z”) and a chimeric receptor including the scFv of 3A5 (“3A5-28z”) were also generated (see FIG. 7A). The cytotoxicity and polyfunctional activity of T cells comprising these receptors were tested. As shown in FIGS. 8 and 9, T cells comprising Meso-282 showed higher cytotoxic activity than T cells comprising 3A5-282, T cells comprising 4h11-282, T cells comprising anti-CD19-chimeric antigen receptor (CAR), and untransduced cells. The secretions of cytokines by these T cells were also measured. As shown in FIG. 10, T cells comprising Meso-28z induced robust antigen-dependent activation as compared to the 3A5-28z.

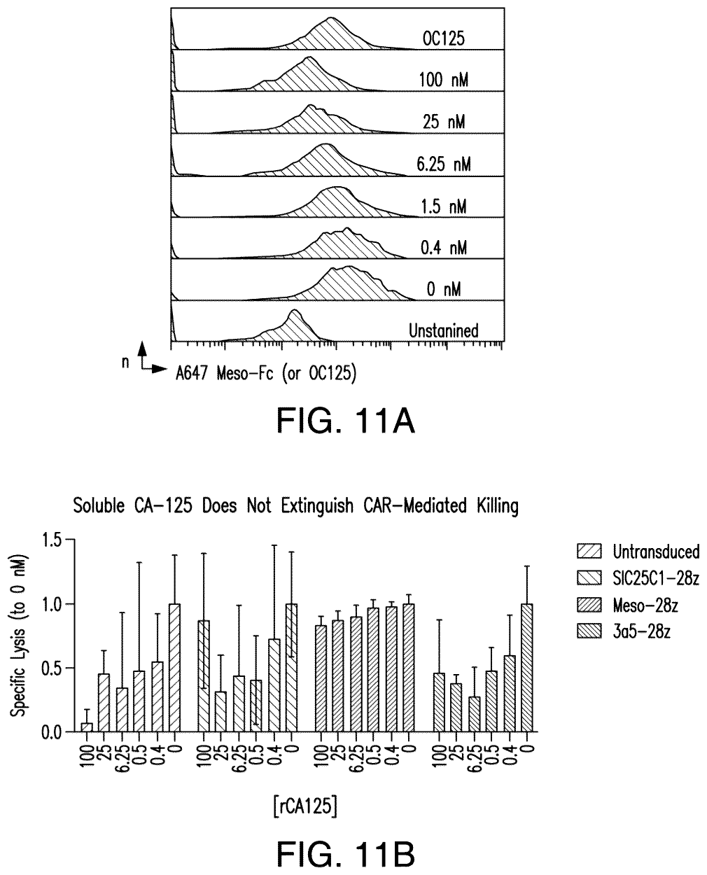

Furthermore, a competitive binding assay was performed using the soluble form of MUC16 (soluble MUC16). As shown in FIGS. 11A and 11B, soluble MUC16 competed with Mesothelin-Fc to bind the MUC16⁺ HT1376 cells in vitro but did not inhibit the cytolytic capacity of Meso-282.

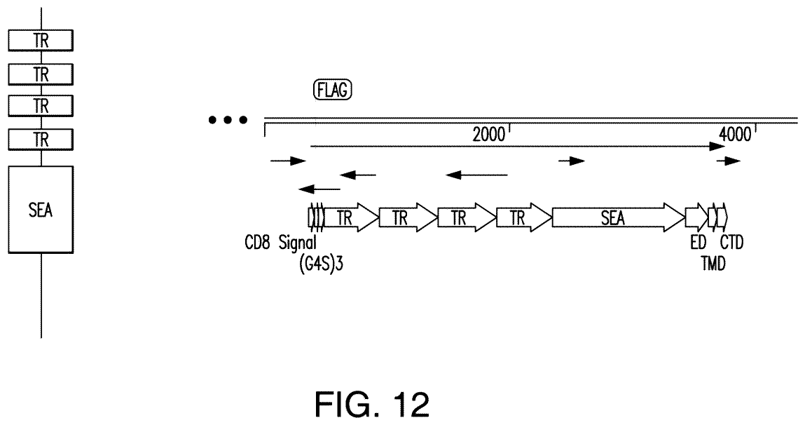

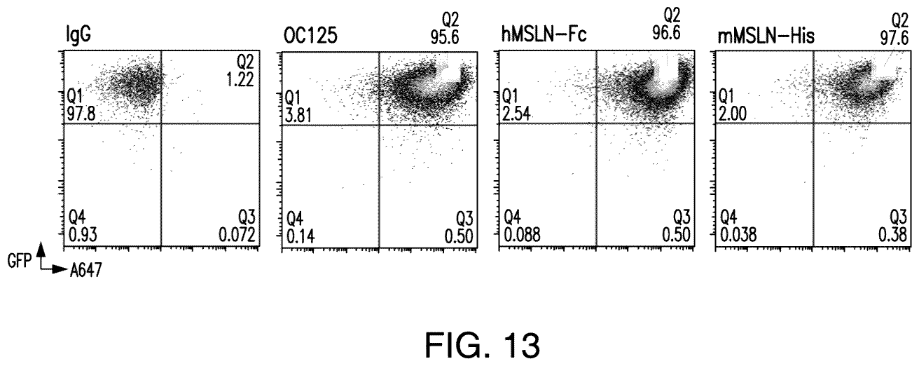

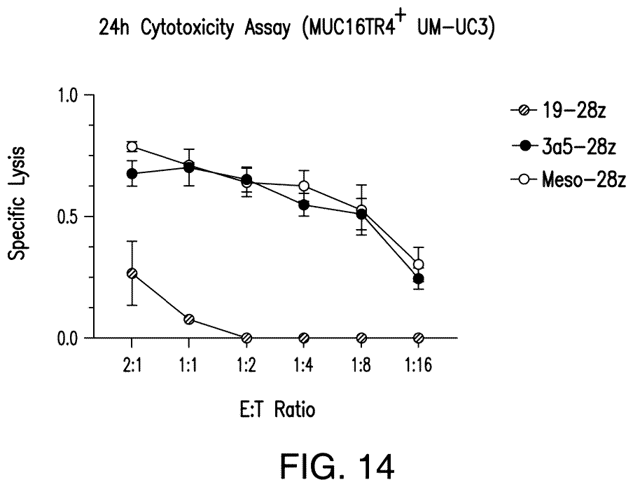

Next, a MUC16 model antigen including four (4) tandem repeats, SEA modules, ectodomain, transmembrane domain and c-terminal domains of MUC16 (see FIG. 12) was developed (designated as “MUC16TR4”). This antigen model was overexpressed in MUC16⁻ UM-UC3 bladder cancer cell line and allowed the recognition of MUC16 by monoclonal antibody OC125 and by mesothelin-Fc proteins (see FIG. 13). As shown in FIG. 14, overexpression of MUC16TR4 on UM-UC3 was sufficient for recognition and killing by 3A5-28z and Meso-28z.

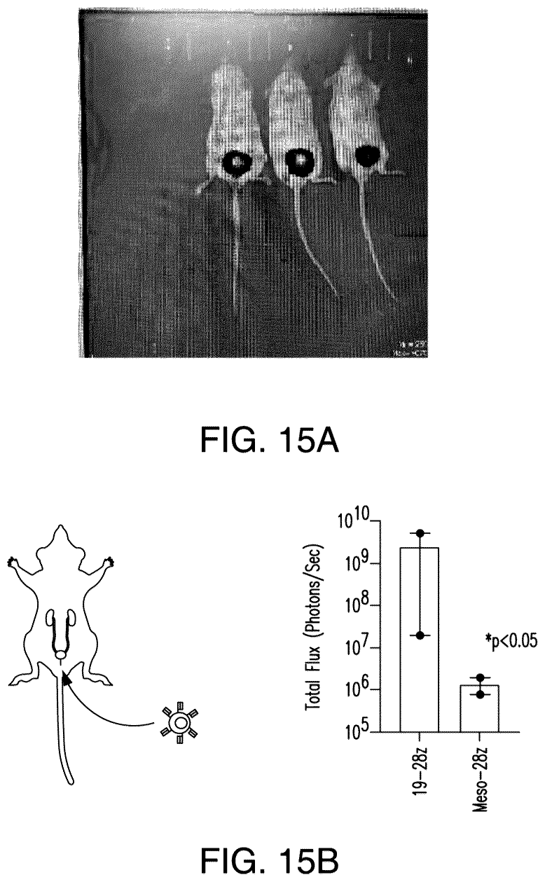

The in vivo activity of Meso-28z was tested. Human orthotopic xenograft of bladder cancer cell lines was generated. See FIG. 15A. As shown in FIG. 15B, intravesically-delivered Meso-28Z CAR T cells decreases tumor burden in an orthotopic human xenograft model.

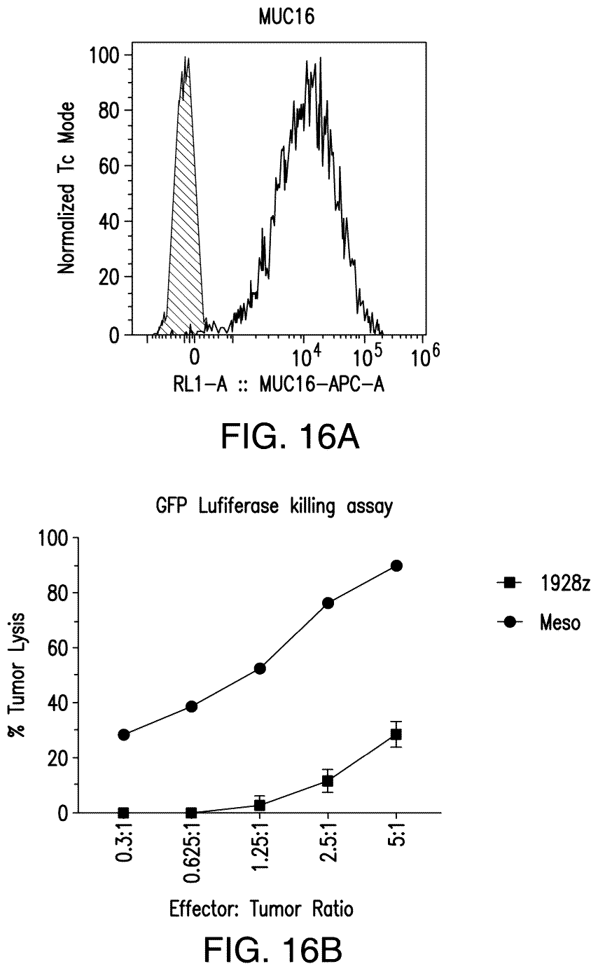

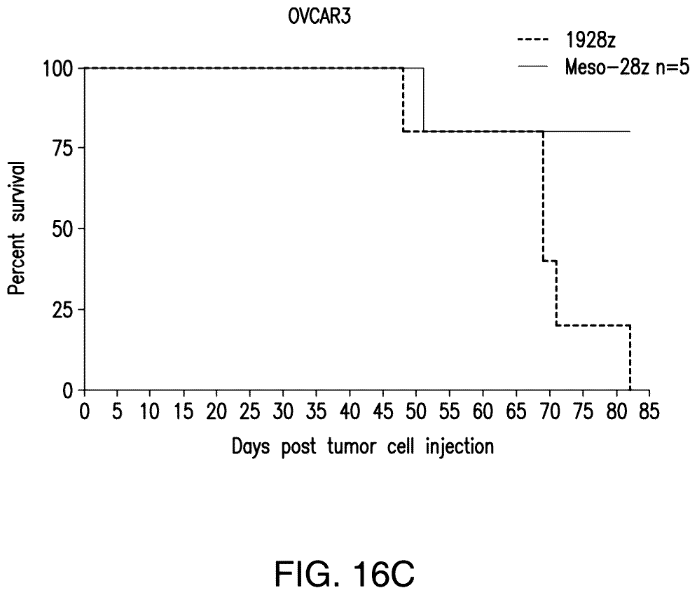

Next, the functions of Meso-28z expressing T cells were tested in ovarian cancer settings. As illustrated in FIG. 16A, ovarian cancer cell line OVCAR3 expresses full length MUC16 on its surface. When co-cultured with Meso-282 T cells, OVCAR3 cells were effectively lysed. In an in vivo xenograft model, NSG mice were inoculated with OVCAR3 cells intra-peritoneally. 3 weeks post tumor injection, 2.5×10⁶ Meso-28z cells were injected intravenously (see FIG. 16B). As demonstrated in FIG. 16C, Meso-28z treatment significantly prolonged animal survival.

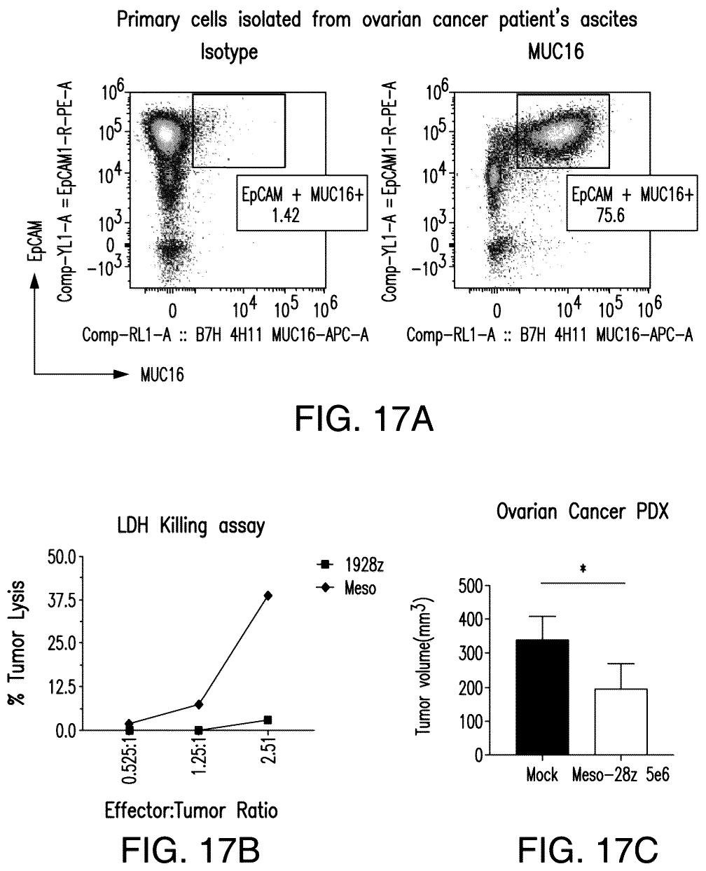

Subsequently, Meso-28z T cell function was evaluated by using primary cancer cells or PDX derived from ovarian cancer patients. As illustrated in FIG. 17A, cancer cells freshly isolated from ovarian cancer patient's ascites expresses MUC16 on cell surface. Importantly, in an LDH killing assay, Meso-28z cells effectively lysed primary tumor cells in comparison to 1928z control T cells (FIG. 17B). In FIG. 17C, ovarian cancer PDX was inoculated into female NSG mice subcutaneously, 5×10⁶ Meso-28z T cells were injected via tail veil. Compared to mock transduced T cells, Meso-28z T cell significantly suppressed tumor growth 7 days post T cell injection, demonstrating its tumor suppression function on a patient derived xenograft model.

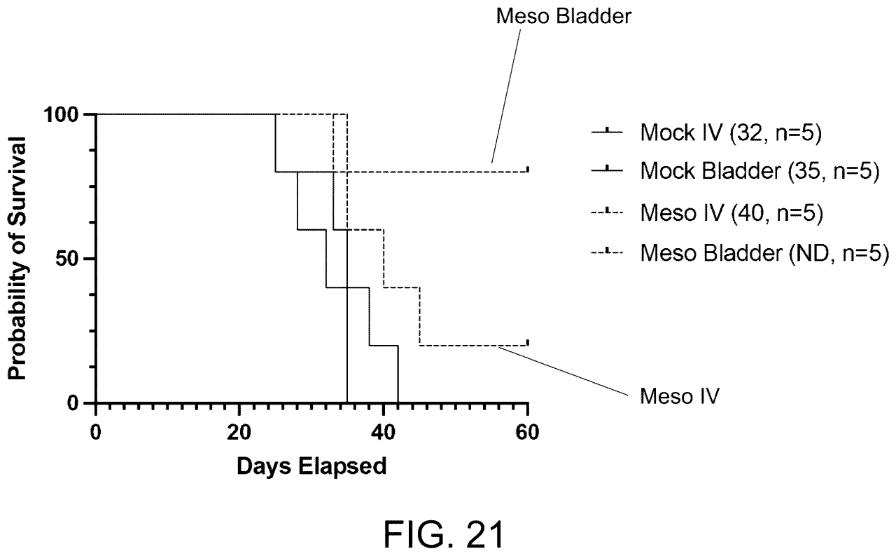

Finally, Meso-28z T cell function was evaluated by using different delivery methods. In these in vivo experiments, T cells comprising Meso-28z were delivered either intravenously (Meso IV) or intravesically (Meso Bladder). As shown in FIG. 21, intravesically-delivered T cells comprising Meso-28z extended survival as compared to intravenous or control T cells in a human orthotopic xenograft model.

### Example 3—T Cells Comprising the Presently Disclosed Chimeric Receptor and the Chemokine Receptor CXCR3

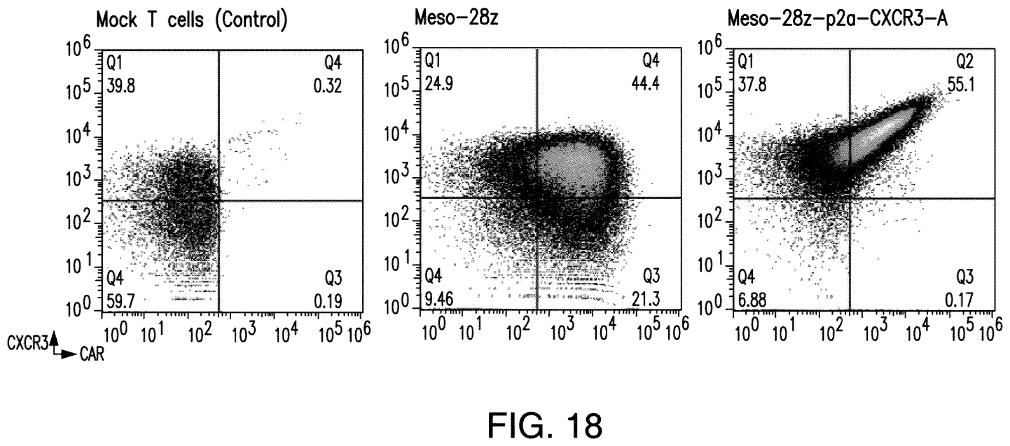

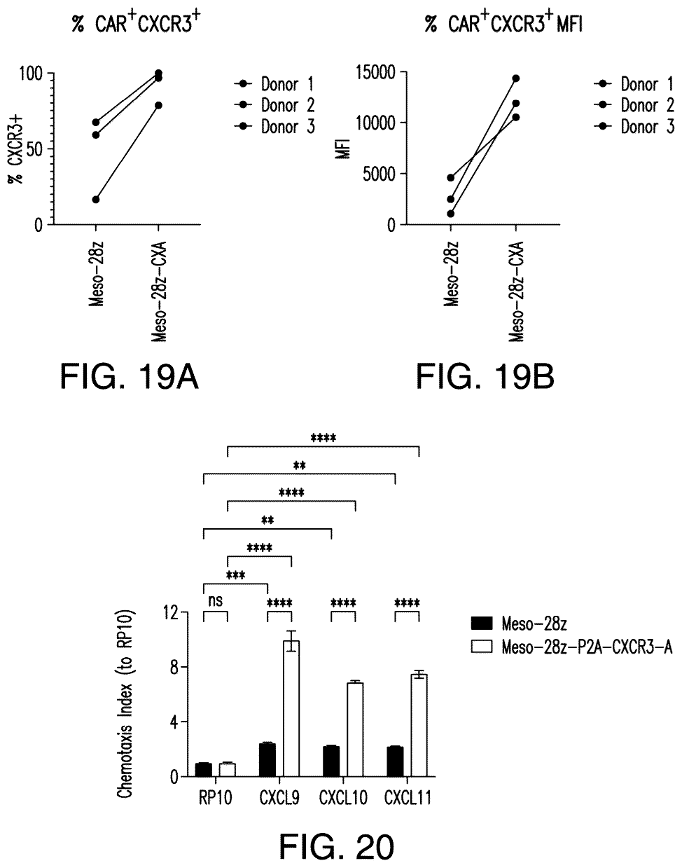

As illustrated in FIG. 18, only a subset of CAR T cells express the chemokine receptor CXCR3, which can be overexpressed in a multicistronic vector. The chemokines CXCL9, CXCL10, and CXCL11 can be up regulated in inflammatory environments and have been reported to be up regulated in many cancers, including bladder cancer. Thus, it was hypothesized that overexpression of CXCR3, the receptor of these chemokines, would enhance the homing, trafficking and expansion of T cells towards the tumor tissue. T cells comprising the presently disclosed chimeric receptors and an exogenous CXCR3 were developed (see FIGS. 7F-7L). These T cells showed a significant increased expression of CXCR3 when compared to the controls (FIGS. 19A and 19B) and exhibited improved chemotaxis properties upon culturing with the chemokines CXCL9, CXCL10, and CXCL11 (FIG. 20). These data demonstrate that overexpression of CXCR3 is involved in the regulation of the trafficking and homing of T cells towards the tumor tissue.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A chimeric receptor, comprising an extracellular domain, a transmembrane domain, and an intracellular signaling domain, wherein the extracellular domain comprises a mesothelin polypeptide that binds to MUC16 and comprises an amino acid sequence that is at least about 80% identical to the amino acid sequence set forth in SEQ ID NO: 1 or a fragment thereof.

2. The chimeric receptor of claim 1, wherein the mesothelin polypeptide comprises or consists of an amino acid sequence that is at least about 80% identical to amino acids 296 to 598 of SEQ ID NO: 1 or a fragment thereof.

3. The chimeric receptor claim 1, wherein the mesothelin polypeptide comprises amino acids 296 to 598 of SEQ ID NO: 1.

4. The chimeric receptor of claim 1, wherein the mesothelin polypeptide comprises an amino acid sequence that is within Region I of human mesothelin.

5. The chimeric receptor of claim 1, wherein the mesothelin polypeptide comprises an amino acid sequence that is within Region IAB of human mesothelin.

6. The chimeric receptor of claim 1, wherein the human mesothelin comprises the amino acid sequence set forth in SEQ ID NO: 1.

7. The chimeric receptor of claim 6, wherein the Region I comprises amino acids 296 to 390 of SEQ ID NO: 1.

8. The chimeric receptor of claim 6, wherein the Region IAB comprises amino acids 296 to 359 of SEQ ID NO: 1.

9. The chimeric receptor of claim 1, wherein the mesothelin polypeptide comprises an amino acid sequence that is at least about 80% identical to the amino acid sequence of amino acids 296 to 359 of SEQ ID NO: 1 or a fragment thereof.

10. The chimeric receptor of claim 1, wherein the mesothelin polypeptide comprises amino acids 296 to 359 of SEQ ID NO: 1.

11. The chimeric receptor of any one of claims 1-11, wherein a signal peptide is covalently joined to the N-terminus of the extracellular domain.

12. The chimeric receptor of claim 1, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM polypeptide, a CTLA-4 polypeptide, a CD27 polypeptide, a CD40 polypeptide, a NKGD polypeptide, a PD-1 polypeptide, a LAG-3 polypeptide, a 2B4 polypeptide, a BTLA polypeptide, or a combination thereof.

13. The chimeric receptor of claim 1, further comprising a hinge/spacer region.

14. The chimeric receptor of claim 1, wherein the intracellular signaling domain comprises a CD3ζ polypeptide.

15. The chimeric receptor of claim 1, wherein the intracellular signaling domain further comprises at least one co-stimulatory signaling region.

16. The chimeric receptor of claim 1, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

17. A cell comprising the chimeric receptor of claim 1.

18. The cell of claim 17, wherein the cell comprises an exogenous chemokine receptor or a fragment thereof or an exogenous IL-18 polypeptide or a fragment thereof.

19. The cell of claim 17, wherein the cell is an immunoresponsive cell.

20. The cell of claim 19, wherein the cell is selected from the group consisting of a T cell, a Natural Killer (NK) cell, a B cell, a monocyte, and a macrophage, a stem cell from which a lymphoid cell may be differentiated, a stem cell from which a myeloid cell may be differentiated, and combinations thereof.

21. The cell of claim 20, wherein the cell is a T cell.

22. A nucleic acid molecule encoding the chimeric receptor of claim 1.

23. A nucleic acid composition comprising a first polynucleotide encoding the chimeric receptor of claim 1 and a second polynucleotide encoding a chemokine receptor or an IL-18 polypeptide.

24. A vector comprising the nucleic acid molecule of claim 22.

25. A lipid nanoparticle comprising the nucleic acid molecule of claim 22.

26. A host cell expressing the nucleic acid molecule of claim 22.

27. A method of reducing tumor burden in a subject having a tumor, increasing or lengthening survival of a subject having a tumor, and/or treating and/or preventing a tumor, comprising administering to the subject the cell of claim 17, wherein the tumor is associated with MUC16.

28. The method of claim 27, wherein the tumor is selected from the group consisting of ovarian cancer, pancreatic cancer, bladder cancer, lung cancer, stomach cancer, endometrial cancer, breast cancer, colorectal cancer, thyroid cancer, and head and neck squamous cell cancers.

29. A method for producing a cell comprising a chimeric receptor of claim 1, comprising introducing into the cell a nucleic acid molecule that encodes the chimeric receptor.

30. A kit for reducing tumor burden in a subject, treating and/or preventing a tumor in a subject, and/or increasing or lengthening survival of a subject having a tumor, comprising the cell of claim 17.
